(19) United States
(12) Patent Application Publication
(10) Pub. No.: US 2025/0218552 A1
(43) Pub. Date: Jul. 3, 2025

(54) Method to Predict Rheology Parameters Using Consistency of a Cement Slurry

(71) Applicant: Halliburton Energy Services, Inc., Houston, TX (US)

(72) Inventors: Xiangyu Liu, Houston, TX (US); Afshin Ahmady, Houston, TX (US); Siva Rama Krishna Jandhyala, Houston, TX (US); K V V N Krishna Babu Yerubandi, Houston, TX (US); Ronnie Glen Morgan, Duncan, TX (US)

(21) Appl. No.: 18/400,162

(22) Filed: Dec. 29, 2023

(51) Int. Cl. G16C 20/30; C09K 8/42

(52) U.S. Cl. CPC G16C 20/30; C09K 8/42

(57) ABSTRACT

A method including providing a consistency model that predicts consistency of a cement slurry design as a function of time (Bc(t)), performing a cementing operation with a cement slurry having a same composition as that of the cement slurry design, and utilizing the consistency model to predict the Bc(t) of the cement slurry during the cementing operation. A system for carrying out the method is also provided.

### FIELD

This application relates to wellbore cementing, and more specifically this application relates to a consistency model that can be utilized, for example, during a cementing operation utilized in the recovery of natural resources from a wellbore penetrating a subterranean formation.

### BACKGROUND

When cement slurry is pumped via a pumping unit down the casing and up via an annulus, the combined effects of friction pressure and hydrostatic pressure must be overcome by the pumping unit. Accordingly, accurately estimating the pressure loss from friction is important in calculating downhole circulation pressure and pump rate during cement placement. These, in turn, govern the displacement efficiencies of the cement job. Conventionally, simulators use temperature-dependent rheology readings, normally measured at surface, bottom hole circulation temperature (BHCT), mainly limited to 190° F., or some midpoint temperature, to calculate frictional and circulating pressures. However, cement slurry rheologies are not only a temperature function but can also be impacted by additional physico-chemical processes, including crystallization, hydration, and gelation during a (e.g., planned or unplanned) shutdown. Due to the use of a time-independent rheological profile for calculations across the entire pumping schedule, a significant error may be introduced in conventional circulation pressure and displacement efficiency calculations.

Accordingly, an ongoing need exists for more accurately estimating pressure loss from friction in calculating downhole circulation pressure and pump rate during cement placement.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1A is a schematic of a method of performing a cementing operation, according to embodiments of this disclosure;

FIG. 1B is a schematic representation of workflow, according to embodiments of this disclosure;

FIG. 2 is a schematic example of predicted consistency vs. observed consistency for a given cement slurry, according to embodiments of this disclosure;

FIG. 3A depicts consistency (Bc) as a function of time during dissolution modeled via a dissolution module [DM], according to embodiments of this disclosure;

FIG. 3B is an example dissolution module [DM], according to embodiments of this disclosure;

FIG. 4A depicts consistency (Bc) as a function of time during thermal thinning modeled via a thermal thinning module [TTM], according to embodiments of this disclosure;

FIG. 4B is an example thermal thinning module [TTM], according to embodiments of this disclosure;

FIG. 5A depicts consistency (Bc) as a function of time during static gelling modeled via a static gelling module [SGM], according to embodiments of this disclosure;

FIG. 5B is an example static gelling module [SGM], according to embodiments of this disclosure;

FIG. 6A depicts consistency (Bc) as a function of time during hydration modeled via a hydration module [HM], according to embodiments of this disclosure;

FIG. 6B is an example hydration module [HM], according to embodiments of this disclosure;

FIG. 7A is an example plot of temperature, actual/observed consistency, and predicted consistency as a function of time, according to embodiments of this disclosure;

FIG. 7B is an example plot of to as a function of time, according to embodiments of this disclosure;

FIG. 7C is an example plot of as a function of time, according to embodiments of this disclosure;

FIG. 8 illustrates a computer that may be used to carry out various steps of or methods according to embodiments of this disclosure;

FIG. 9 is a schematic of surface equipment that can be utilized in the placement of a cement slurry, according to embodiments of this disclosure; and

FIG. 10 is a schematic of the placement of a cement slurry into a subterranean formation, according to embodiments of the disclosure.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

It is to be understood that “subterranean formation” encompasses both areas below exposed earth and areas below earth covered by water such as ocean or fresh water. Herein in the disclosure, “top” means the well at the surface (e.g., at the wellhead which may be located on dry land or below water, e.g., a subsea wellhead), and the direction along a wellbore towards the well surface is referred to as “up”; “bottom” means the end of the wellbore away from the surface, and the direction along a wellbore away from the wellbore surface is referred to as “down.” For example, in a horizontal wellbore, two locations may be at the same level (i.e., depth within a subterranean formation), the location closer to the well surface (by comparing the lengths along the wellbore from the wellbore surface to the locations) is referred to as “above” the other location, the location farther away from the well surface (by comparing the lengths along the wellbore from the wellbore surface to the locations) is referred to as “below” or “lower than” the other location.

As noted hereinabove, when a cement slurry is pumped downhole (e.g., down a casing and up in an annulus between the casing and a wellbore wall or vice versa), the combined effects of hydrostatic pressure and friction pressure must be overcome by the pumping unit. Accurately estimating the pressure loss from friction is important in calculating downhole circulation pressure and pump rate during the placement of the cement. The circulation pressure and pump rate are key parameters that govern the displacement efficiencies of the cement job. Conventional simulators employ temperature-dependent rheology readings (e.g., measured at surface, BHCT, mainly limited to 190° F., or some midpoint temperature) to calculate frictional and circulating pressures. However, as noted hereinabove, cement slurry rheologies are not only a temperature function but can also be impacted by additional physico-chemical processes, such as crystallization, hydration, and gelation during a planned (and/or unplanned) shutdown. Due to the use of a time-independent rheological profile for calculations across the entire pumping schedule, a significant error can be introduced in circulation pressure and displacement efficiency calculations.

The present disclosure provides systems and methods that derive time-dependent rheology using a thickening time curve. The derived time-dependent rheology is subsequently utilized to more accurately predict circulating pressures and displacement efficiencies relative to conventional methods that use time-independent rheology profiles. The thickening time curve is systematically parsed via the herein disclosed system and method, to derive the time-dependent rheology. In embodiments, the following steps can be utilized to systematically parse the thickening time curve.

A four-component model is utilized to predict the physico-chemical phenomena that are part of a typical thickening time curve:
(a) a Dissolution Module (DM) is utilized to pattern the dissolution of slurry components (e.g., during an early “temperature ramping” stage) within a short time after mixing, for example, hydration of polymers, further wetting of bulk blended particles, and further hydration of additives;
(b) a Thermal Thinning Module (TTM) is utilized to model thermal thinning either during or after the dissolution phase, when the slurry rheology (measured via the Beardon consistency units (Bc)) may experience a decline, commonly referred to as “thermal thinning”. It is noted that it is common to see an initial rise in Bc vs. time during the early temperature ramping, such as (but not limited to) the 80 to 120° F. range when dissolution and thermal thinning regimes overlap;
(c) a Static Gelling Module [SGM](also referred to as a shutdown gelling module) is utilized to model a static period (e.g., a period(s) during which the consistometer rotation is turned off to simulate planned shutdowns during the cementing job). During this time of static gelling, certain cement slurries can exhibit preliminary gelling or very low levels of localized cement hydration occurring due to the “static state” during shutdown; and
(d) a hydration of the cementitious particles module or Hydration Module [HM] which models a period during which the cement hydrates. Hydration can be modeled via a hydration time period from when the consistometer is restarted and resumes rotation of 150 RPM until 70 Bc is reached.
   It should be noted that the herein proposed modules and subsequent rheological models can be applicable to a different rotations speeds before and after shut down, and that the proposed invention is not limited to 70 Bc being the final Bc reading.

Once the above “Four Modular Model” (also referred to herein as a consistency model) is defined for a given slurry, thus providing a method to predict Bc vs. time for any given real-time job temperature-time (T-t), pressure-time (P-t), and pumping schedule, a novel method is further utilized for transforming the predicted Bc-t data from above, into Generalized Herschel-Bulkley Rheology parameters, thus facilitating the ability to predict (e.g., changes in) equivalent circulating density (ECD) and pumping pressure. SPE publication 82415, entitled “Improved Rheology Model and Hydraulics Analysis for Tomorrow's Wellbore Fluid Applications”, provides a description of the Generalized Herschel-Bulkley Rheological model, and is hereby incorporated herein in its entirety for purposes not contrary to this disclosure.

The novel method disclosed herein eliminates the need to calibrate the Bc reading for each HPHT consistometer for any given cement slurry. In conventional methods, obtaining the apparent viscosity of cement from consistency reading involves multiplying Bc reading by a conversion constant, potentially incorporating any errors that exist in the consistency measurement. The novel method disclosed herein offers a more error-resistant methodology by conducting an accurate rheology test at a reference point; rheology parameters are then scaled based on the changes in Bc reading over time using Equations 7 and 8 described hereinbelow.

A method of this disclosure can thus comprise conducting a typical API thickening time (TT) test with an HPHT (high pressure high temperature) consistometer to measure Bc (torque that relates to rheology) vs. time for a given expected wellbore temperature-time (T-t) and pressure-time (P-t) schedule, while the HPHT consistometer is rotating at a constant speed (e.g., of 150 RPM). It should be noted that the herein proposed modules and subsequent rheological models can be applicable to a different rotations speeds, other than just 150 RPM which is convention, before and after shut down, and that the proposed invention is not limited to 70 Bc being the final Bc reading.

Bc can be correlated to viscosity at a known shear rate of approximately 56 1/s (56 s⁻¹) for the conventional consistometer cup and 38 1/s (38 s⁻¹) shear rate for the “thixotropic cup” assuming rotation speed is constant at 150 RPM. The typical Bc TT test can comprise, consist of, or consist essentially of (a) loading the cement slurry into either a “conventional HPHT cup” or a “thixotropic cup”; (b) starting the rotation of the cup at a constant speed (e.g., of 150 RPM) and continuously recording the “Bc” reading, which is a known torque value for the entire testing regimen; (c) ramping to BHCT (or another selected temperature) and selected pressure in a given period; (d) holding the cement slurry at the selected temperature (e.g., BHCT) and selected pressure for a selected period; (e) turning off the rotation for a specified time, called shutdown time; (f) turning the rotation back on after the specified shutdown time; (g) continuing to measure the Bc vs. time until the Bc value reaches final Bc (e.g., 70Bc), at which time the test can be terminated; (h) correlating the Bc to viscosity at a known shear rate (e.g., of approximately 56 1/s, assuming rotation speed is constant at 150 RPM). It is again noted that the herein proposed modules and subsequent rheological models can be applicable to a different rotations speeds, other than just 150 RPM which is convention, before and after shut down, and that the proposed invention is not limited to 70 Bc being the final Bc reading.

Conventionally, the primary value of a TT test is to ascertain the time from starting the test until 70 Bc is reached, which is the value at which a cement slurry is deemed “unpumpable”. This TT test is a tool that can be used by a drilling optimization specialist (DOS) for tailoring the time delay to facilitate placement of the cement to the targeted top of cement (TOC), as well as to provide additional contingency time by using retarder chemistry.

As more cementing jobs are being completed in more complex well geometries and reservoirs, it would be very beneficial to have a method that would allow the making of “real-time predictions” of the impact of unplanned shutdown times or slower/faster pumping rates that alter the planned T-t and P-t job history that was utilized for the TT testing. The herein disclosed systems and methods provide for such real-time predictions, enabling the prediction of changes to the Bc-t curve, when T-t and P-t are different from those used in the HPHT consistometer TT testing; transforming of the Bc-t predictions into rheological properties of cement as the properties change over time (“rheology vs. time”) during cement placement; and converting of the so obtained “rheology vs. time” into friction pressure changes in the well bore, thus providing for “Real Time—On the Job” prediction of the impact of unplanned pumping schedule changes on ECD and surface pressure.

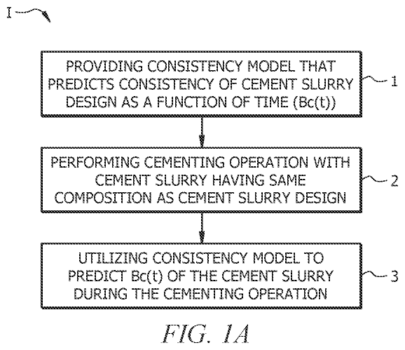

As detailed further hereinbelow with reference to FIGS. 1-6, the method of this disclosure can comprise Part 1, in which, for a given cement slurry, the Four Component Modules noted above (e.g., [HM], [TTM], [SGM], [HM]) are utilized to predict the Bc vs. time curve, and Part II (indicated as 20 in FIG. 1), in which the Bc-t curve is transformed to Generalized Herschel Bulkley (GHB) parameters vs time. Existing algorithms (e.g., in iCem® Service, available from Halliburton Energy Services of Houston, Texas) can be utilized to calculated ECD and frictional pressure. Steps (of Part 2) can include (1) conducting the cement rheology test using a rotational viscometer in accordance with API RP 10B-2 at a reference temperature, which, as noted above, can be room temperature, BHCT or any middle point; (2) calculating the rheological parameters (tauₒ, mu_{∞}, m and n) applying a GHB rheological model (as displayed in Equation (6) hereinbelow); (3) for Bc vs. time data from 6/Part I, from time 0 until Bc reaches 70, compute adjusted_tau,o and adjusted_mu,∞ (e.g., using Equation (7) and Equation (8) provided hereinbelow), while m and n remain constant; and (4) utilizing the adjusted rheological parameters for subsequent hydraulic calculations using existing algorithms (e.g., in iCem® Service or another software or analysis).

Given a TT test result, the herein disclosed consistency model enables prediction of a new Bc vs. time curve for the (e.g., actual) cementing job, thus facilitating the prediction of the GHB parameters. The FMM comprises:

$$Bc ( t ) = B {c}_{i} \times [ DM ] \times [ TTM ] \times [ SGM ] \times [ HM ] , \qquad Eq . ( 1 )$$

where Bc(t) is Bc at time t, Bcᵢ is initial Bc, [DM] is the dissolution module, [TTM] is the thermal thinning module, [SGM] is the static gelling module, and [HM] is the hydration module. The four component modules detailed further hereinbelow (e.g., [DM], [TTM], [SGM], [HM]) thus enable the prediction of the Bc vs. time curve.

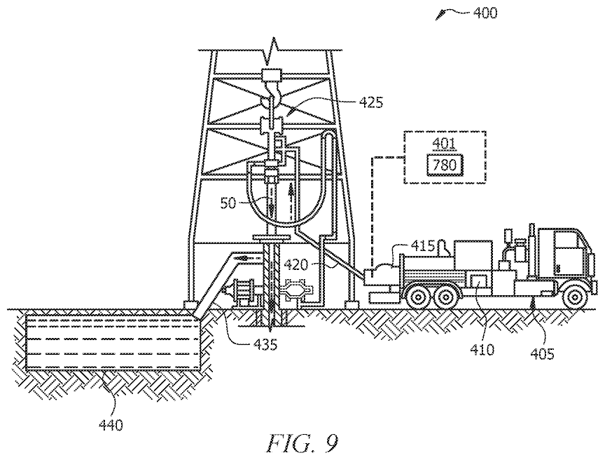

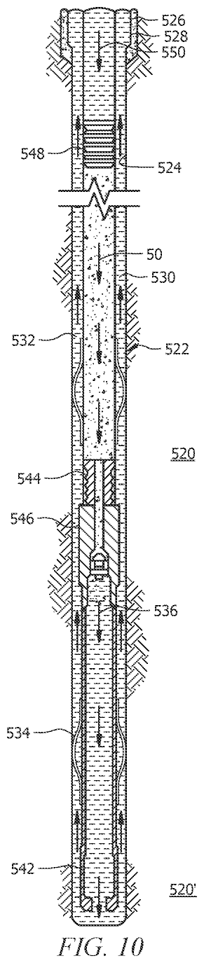

FIG. 1 is a schematic of a method of performing a cementing operation according to embodiments of this disclosure. Method I comprises: providing, at 1, a consistency model (e.g., a four module model (FMM)) that predicts consistency of a cement slurry design as a function of time (Bc(t)); performing, at 2, a cementing operation with a cement slurry 50 (FIG. 9 and FIG. 10) having a same composition as that of the cement slurry design; and utilizing, at 3, the consistency model to predict the Bc(t) of the cement slurry 50 during the cementing operation.

With reference to FIG. 1B, which provides a workflow of a method II of this disclosure for calculating ECD and pressure using time dependent GHB parameters calculated from TT test, providing the consistency model at 1 can comprise: designing or providing the cement slurry at 5, obtaining, at 6, consistency versus time (Bc(t)) data for a sample of the cement slurry design from an initial time (to) of mixing sample of the cement slurry design to a thickening time (t_{TT}) at which the consistency of the sample reaches 70 Bc (Beardon consistency units); and, via Part 1, utilizing the consistency versus time data to determine coefficients utilized by the consistency model to predict the consistency of the cement slurry design as a function of time. Utilizing the consistency versus time data obtained at 6 to determine the coefficients can comprise statistical regression until a predicted Bc(t) adequately simulates/models the Bc(t) data (e.g., to within a desired delta, such as less than 5, 4, 3, 2, or 1%, between the predicted Bc(t) and the measured/actual Bc(t)).

The consistency model predicts the consistency as a function of time (Bc(t)) generated at 15 via: a dissolution module [DM] that models the consistency behavior during dissolution of particles, if present, from the initial time (t₀) to a dissolution time (t_{d}) at an end of dissolution; a thermal thinning module [TTM] that models thermal thinning, if present, during initial ramping of the temperature; a static gelling module [SGM] that models consistency behavior of the cement slurry during a shut down, if present, from an initial shutdown time (t_{sd, start}) to an end of the shutdown (t_{sd, end}); and a hydration module [HM] that models the consistency behavior during hydration of cementitious particles from an initial hydration time (tₕₒ) to the thickening time (t_{TT}). Accordingly, Part I, indicated at 10, of the method II can comprise calculating, as indicated at 11, the dissolution module [DM] using initial Bc reading Bcᵢ, calculating, as indicated at 12, the thermal thinning module [TTM] using temperature profile, calculating, as indicated at 13, the static gelling module [SGM] using Bc data before and after planned shutdown, and calculating, as indicated at 14, the hydration module [HM] using Bc data from the beginning of hydration until Bc equals 70 Bc. As indicated at 13A, calculating [SGM] at 13 enables calculation of predicted Bc if shutdown schedule deviates from planned. As indicated at 15, Part I (10) of the method II comprises generating the predicted Bc vs. time curve, wherein, as noted in Equation (1) hereinabove: Bc(t)=Bc(i)[DM][TTM][SGM][HM], wherein Bc(i) is the initial consistency at t₀.

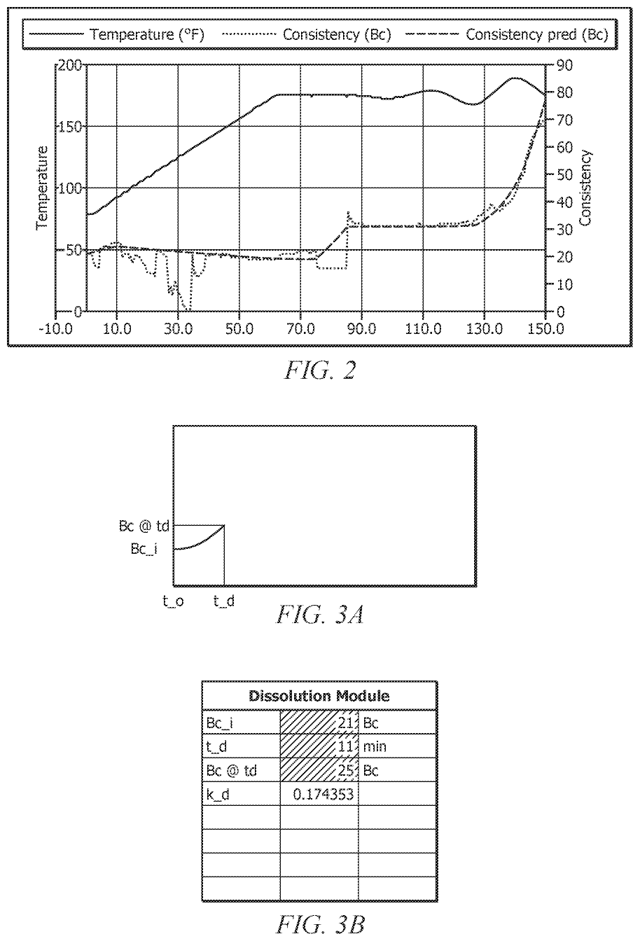

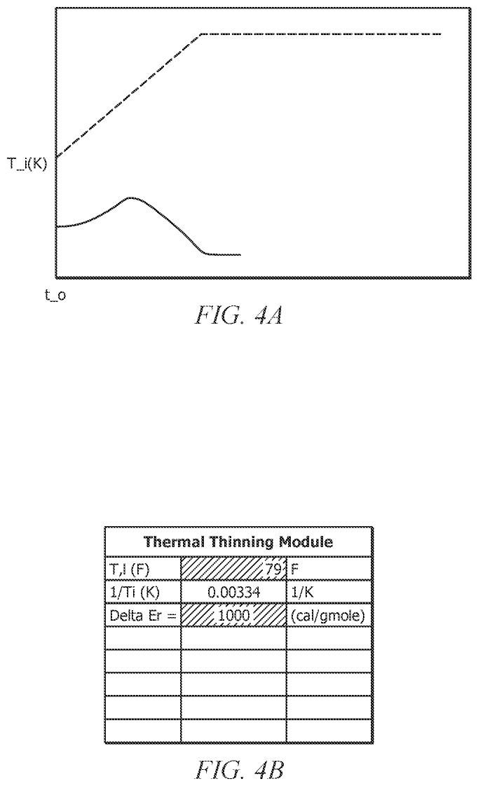

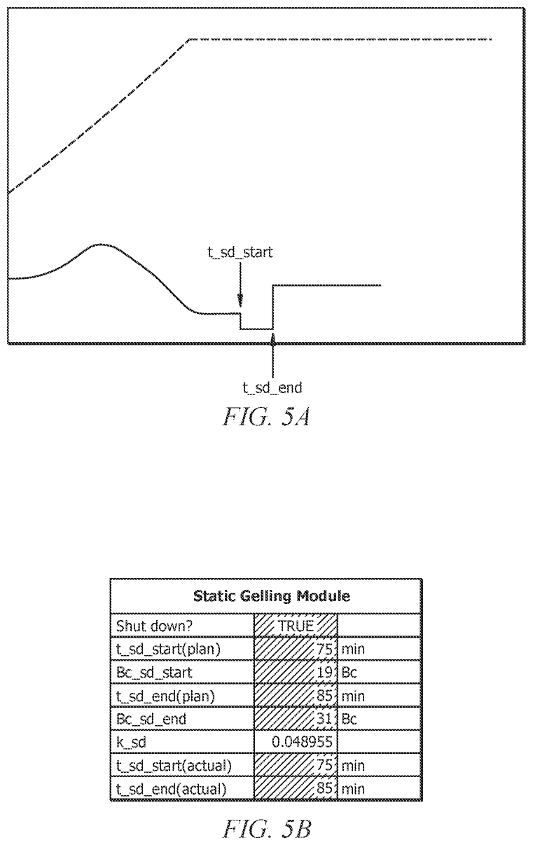

A method of providing a consistency model that predicts consistency of cement slurry design as a function of time (Bc(t)) at 1 can thus comprise: obtaining, at 6, consistency versus time (Bc(t)) data from a thickening time (TT) test for a cement slurry design from an initial time (t₀) of mixing the cement slurry design to a thickening time (t_{TT}) at which the consistency reaches 70 Bc (Beardon consistency units); and utilizing the consistency versus time data to determine parameters for a consistency model to predict the consistency of the cement slurry design as a function of time at 15. Utilizing the consistency versus time data to determine the parameters for the consistency model employs steps 11-14, as noted above and detailed further hereinbelow. The consistency model can predict the consistency of the cement slurry design as a function of time (Bc(t)) via: the dissolution module [DM] that models the consistency behavior of the cement slurry design during dissolution of particles therein from the initial time (to) to a dissolution time (t_{d}) at an end of dissolution (as described further hereinbelow with reference to FIG. 3A and FIG. 3B); the thermal thinning module [TTM] that models thermal thinning, if present, of the cement slurry design from during initial ramping of the temperature during the obtaining of the consistency versus time (Bc(t)) data for the cement slurry design (as detailed further hereinbelow with reference to FIG. 4A and FIG. 4B); the static gelling module [SGM] that models consistency behavior of the cement slurry design during a shut down from a start of the shutdown (t_{sd, start}) to an end of the shutdown (t_{sd, end}) (as detailed further hereinbelow with reference to FIG. 5A and FIG. 5B); and the hydration module [HM] that models the consistency behavior of the cement slurry design during hydration of cementitious particles therein from an initial hydration time (tₕₒ) to the thickening time (t_{TT}) at which the consistency reaches 70 Bc. As noted above, at 15, the predicted Bc vs. time curve is generated as per Equation (1) above: Bc(t)=Bc(i)[DM][TTM][SGM][HM], wherein Bc(i) is the initial consistency of the cement slurry deign at to.

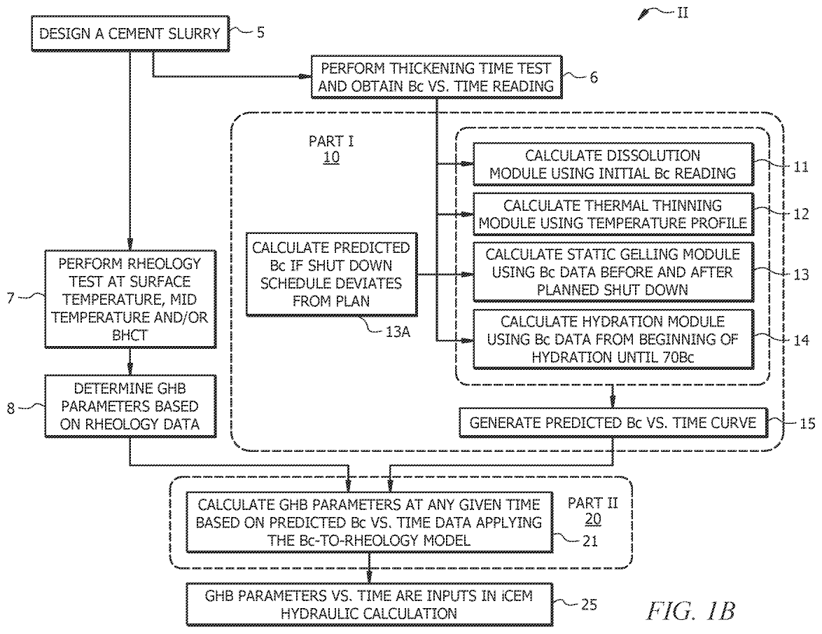

With reference to FIG. 2, which is a schematic example of predicted consistency vs. observed consistency for a given cement slurry, according to embodiments of this disclosure, Part I of the method utilizes the four modules to provide a predicted Bc(t) that acceptably predicts the measured Bc(t). Statistical regression analysis can be utilized to match the measured consistency with the predicted consistency.

FIG. 3A depicts consistency (Bc) as a function of time during dissolution modeled via a dissolution module [DM], according to embodiments of this disclosure; and FIG. 3B is an example dissolution module [DM] computer display, according to embodiments of this disclosure;

In embodiments, the dissolution module [DM] is active if t_{d}>0, wherein:

if t_{d}=0, [DM]=1.0, Eq. (2A)

when t<t_{d}, [DM]=e\hat{ }(k_{d}t/t_{d}), Eq. (2B)

when t≥t_{d}, [DM]=[DM] at t=t_{d}, Eq. (2C)

wherein to is the initial time of mixing, at the beginning of the TT test, Bcᵢ is the initial consistency (e.g., Bc reading) at the beginning of the TT test, and t_{d} is the dissolution time at an end of dissolution (e.g., when a maximum Bc reading, Bc_{td}, is obtained). Other mathematical representations of the dissolution are within the scope of this disclosure, and the above representation is provided merely as an example.

FIG. 4A depicts consistency (Bc) as a function of time during thermal thinning modeled via a thermal thinning module [TTM], according to embodiments of this disclosure; and FIG. 4B is an example thermal thinning module [TTM], according to embodiments of this disclosure.

In embodiments, the [TTM] module is only active if thermal thinning behavior is present, and

$$[ TTM ] = e \hat{} ( ( ( Delta E ) / R ) ( ( 1 / T ) - ( 1 / {T}_{i} ) ) , \qquad Eq . ( 3 )$$

wherein Delta E is an activation energy for thermal thinning (e.g., typically ranging from 0 to 5,000 cal/gmol), R is the universal gas constant (e.g., 1.986 cal/gmol-K), Tᵢ is a temperature at the beginning of the TT test in Kelvin, and T is temperature at any given time t in Kelvin. Other mathematical representations of the thermal thinning are within the scope of this disclosure, and the above representation is provided merely as an example.

FIG. 5A depicts consistency (Bc) as a function of time during static gelling modeled via a static gelling module [SGM], according to embodiments of this disclosure; and FIG. 5B is an example static gelling module [SGM], according to embodiments of this disclosure.

In embodiments, the [SGM] module is only active if there is a shut down (e.g., Shut Down is True), and

when t≥t_{sd,start}, [SGM]=e\hat{ }((k_{sd}(t_{sd,end}−t_{sd,start}))), Eq. (4)

wherein t_{sd,start} is a time at which rotation speed is stopped at the start of shut down, t_{sd,end} is a time at which rotation speed is resumed (e.g., to 150 RPM) after the shut down, and k_{sd} is a coefficient of static gelling reaction. The [SGM] enables prediction by the consistency model of an adjusted consistency at the end of the shut down (Bc_{sd,end}) if a shut down schedule changes during an actual cementing operation. Other mathematical representations of static gelling are within the scope of this disclosure, and the above representation is provided merely as an example.

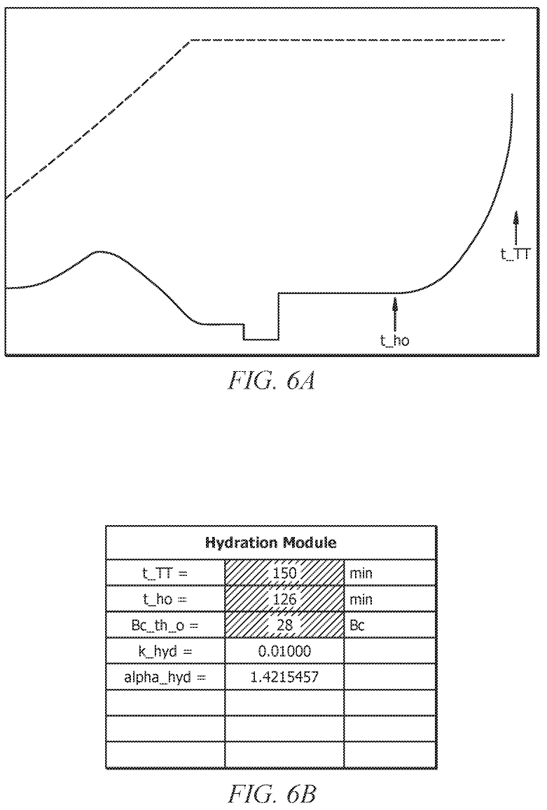

FIG. 6A depicts consistency (Bc) as a function of time during hydration modeled via a hydration module [HM], according to embodiments of this disclosure; and FIG. 6B is an example hydration module [HM], according to embodiments of this disclosure.

In embodiments,

$$[ HM ] = e \hat{} ( ( {k}_{h y d} ) {( t - {t}_{h 0} )}^{α} ) from t = {t}_{h 0} to t = {t}_{T T} , \qquad Eq . ( 5 )$$

wherein t_{TT}=thickening time to 70Bc, tₕₒ is a time when hydration begins, t=time, α (e.g., alpha_hyd in FIG. 6B) is a pseudo reaction order coefficient, and k_{hyd} is a hydration coefficient. Other mathematical representations of hydration are within the scope of this disclosure, and the above representation is provided merely as an example.

As noted hereinabove, in Part I, utilizing the consistency versus time data (e.g., obtained at 6) to determine parameters for the consistency model to predict (e.g., at 15) the consistency of the cement slurry design as the function of time can comprise utilizing statistical regression to determine the parameters for the consistency model.

A method of this disclosure can thus include performing the TT test to obtain the consistency versus time (Bc(t)) data for the cement slurry from the initial time (t₀) of mixing the cement slurry design to the thickening time (t_{TT}) at which the consistency reaches 70 Bc (Beardon consistency units). As noted hereinabove, performing the TT (thickening time) test can comprise: loading a sample of the cement slurry design into a cup (e.g., either a “conventional HPHT cup” or a “thixotropic cup”); starting rotation of the cup at a constant speed (e.g., of 150 RPM) at time t₀; ramping the temperature from an initial temperature Tᵢ to a second temperature (e.g., BHCT) and a selected pressure in a temperature ramping time period; holding the sample at the second temperature (e.g., BHCT) and the selected pressure for a holding period; turning off the rotation for a shutdown time from t_{sd,start} to t_{sd,end}; turning the rotation back on at t_{sd,end} (e.g., after the shutdown time); continuing to measure the Bc vs. time of the sample until the Bc value reaches 70, at time t_{TT}; and continuously recording the consistency (or “Bc”) reading (e.g., also known torque value) from the starting of the rotation of the cup at time to until time t_{TT}. Other methods of performing a TT test are within the scope of this disclosure, and the heretofore described TT test method is provided merely by way of example.

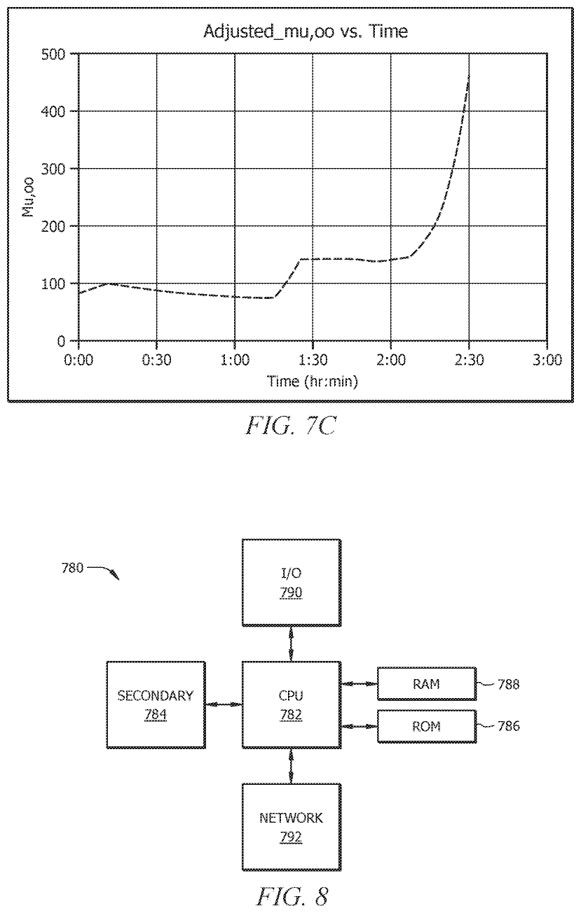

As noted at 3 in FIG. 1A, method I can include utilizing the consistency model during a cementing operation with a cement slurry having the same composition as the cement slurry design to predict Bc(t) (e.g., and thus provide an actual time t_{TT} at which the cement slurry will reach 70 Bc) during the cementing operation. Utilizing the consistency model during the cementing operation with the cement slurry to predict the actual time t_{TT} at which the cement slurry will reach 70 Bc during the cementing operation can further comprise: adjusting a time, a temperature, a consistency reading, or a combination thereof utilized by the consistency model with an actual time, an actual temperature, an actual consistency reading, or a combination thereof obtained during the cementing operation when the actual time, the actual temperature, the actual consistency reading, or the combination thereof obtained during the cementing operation is different from the time, the temperature, the consistency reading, or the combination thereof utilized to determine the parameters for the consistency model. The actual readings obtained during the cementing operation can be input, for example, via a user interface on a computer, such as described hereinbelow with reference to FIG. 8. Representative [DM], [TTM], [SGM], and [HM] modules are shown in FIG. 3B, FIG. 4B, FIG. 5B, and FIG. 6B.

A method of this disclosure can further comprise utilizing the Bc(t) predicted during the cementing operation at 3 to determine how one or more rheological properties of the cement slurry will change over time during the cementing operation. For example, the determined changes in the one or more rheological properties with time can be utilized to determine a hydraulic parameter of the cementing operation, such as an equivalent circulation density (ECD), a surface pressure, or a combination thereof.

With reference to FIG. 1B, utilizing the Bc(t) predicted during the cementing operation to determine how the one or more rheological properties of the cement slurry 50 will change over time during the cementing operation can further include: as indicated at 7, performing a rheology test at a reference temperature (e.g., a surface temperature, a mid temperature, and/or BHCT) to measure rheology data; as indicated at 8, determining GHB parameters based on the measured rheology data; as indicated at 21, calculating GHB parameters as a function of time based on the predicted Bc(t) during the cementing operation, applying the Bc-to-rheology model in Equations 7 and 8; and, as indicated at 25, utilizing the GHB parameters as a function of time in the hydraulic calculation.

Performing the rheology test at the reference temperature (e.g., surface temperature, a middle point temperature, and/or BHCT) to measure rheology data at 7 can comprise conducting the rheology test using a rotational viscometer in accordance with API RP 10B-2 at the reference temperature.

Utilizing the Bc(t) predicted to determine how the one or more rheological properties of the cement slurry will change over time during the cementing operation can further comprise: for the cement slurry, calculating the rheological parameters (tau₀ (τ₀), mu_{∞,0}, m, and n, e.g., at 80° F.) applying a GHB rheological model (e.g., as displayed in Equation (6) below); from Bc(t), from time to until Bc reaches 70 at t_{TT}, calculating adjusted rheological parameters by computing tau₀ and mu_{∞,0} as a function of time, while m and n remain constant; (e.g., as in Equations 7 and 8 below); and utilizing the adjusted rheological parameters for subsequent hydraulic calculations (e.g., using existing algorithms in iCem® Service).

The Bc-to-rheology model can thus be utilized to transform the Bc-t curve to GHB parameters vs. time, more specifically tau,o vs. time and mu,∞ vs. time. Subsequently, existing algorithms (e.g., in iCem® Service or another program) can utilize adjusted tau,o and mu,∞, along with other job parameters, to calculate hydraulic friction and ECD.

Calculating the rheological parameters (tau₀ (τ₀), mu_{∞,0}, m, and n, e.g., at 80° F.) applying the GHB rheological model can comprises using the Equation (6):

$${( \frac{τ}{{τ}_{ref}} )}^{m} = {( \frac{{τ}_{o}}{{τ}_{ref}} )}^{m} + {( \frac{{μ}_{∞} \dot{γ}}{{τ}_{ref}} )}^{n} \qquad Eq . ( 6 )$$

wherein, for Bc vs. time data, until 70Bc, computing tau₀ and mu_{∞}, while m and n remain constant comprises using Equation (7) and Equation (8):

$${( \frac{{τ}_{o , i + 1}}{{τ}_{o , i}} )}^{m} = \frac{B {c}_{i + 1}}{B {c}_{i}} \qquad Eq . ( 7 )$$

$${( \frac{{u}_{∞ , i + 1}}{{u}_{∞ , i}} )}^{n} = \frac{B {c}_{i + 1}}{B {c}_{i}} \qquad Eq . ( 8 )$$

wherein i+1 denotes the timestamp immediately succeeding t_i. For Bc_i, tau₀_i and mu_{∞}_i, the subscript indicates the values corresponding to the time stamp i, and Bc_i+1, tau₀_i+1 and mu_{∞}_i represent the values at the subsequent time stamp i+1.

Tau,o (τ₀) and mu_∞ (μ_{∞}) are GHB parameters in Equation (6). Tau_0 is the shear stress. Mu_∞ is High Shear Rate AVIS Plateau. AVIS is the Apparent Viscosity at a given Shear rate for the GHB model. Gamma (γ) is the shear rate.

Utilizing the adjusted rheological parameters for subsequent hydraulic calculations can further comprise: computing the apparent viscosity using adjusted values of Tau,o vs. time and Mu,∞ vs. time and m and n along with other job parameters including fluid density, rate, well bore geometry; computing hydraulic friction based on planned or real time events during the job.

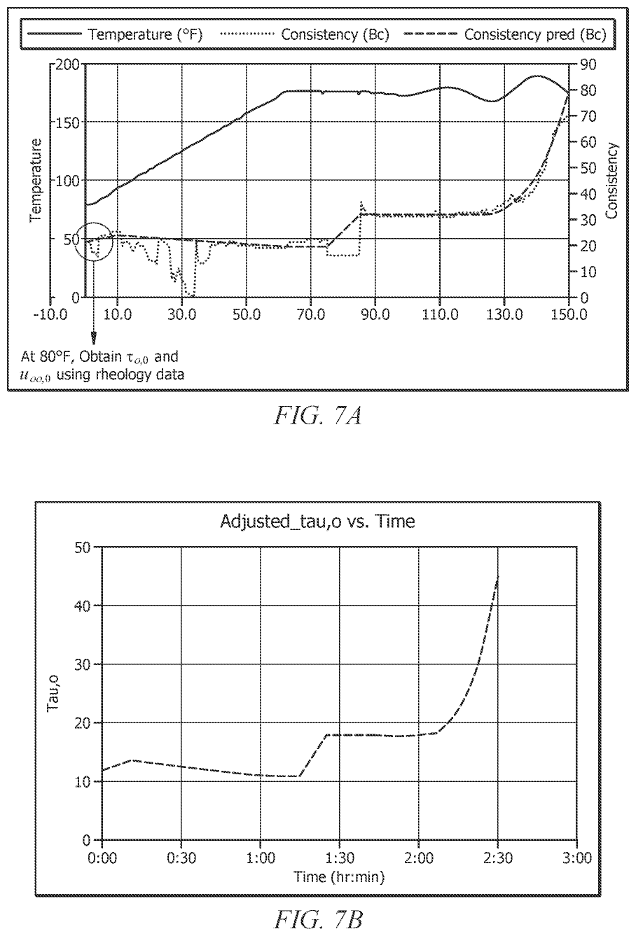

FIG. 7A is an example plot of temperature, actual/observed consistency, and predicted consistency as a function of time, according to embodiments of this disclosure, indicating how to and can be obtained from data obtained at 7; FIG. 7B is an example plot of adjusted to as a function of time, according to embodiments of this disclosure; and FIG. 7C is an example plot of adjusted as a function of time, according to embodiments of this disclosure.

In embodiments, a method of designing a cement placement job comprises: predicting a hydraulic parameter of a cementing operation utilizing adjusted time dependent rheological model parameters, obtaining a time dependent rheological model parameter using a predicted time dependent Bc curve, and predicting the time dependent Bc curve using models that describe at least one of a parameter selected from the group consisting of dissolution, thermal thinning, static gelling, and hydration processes in cement. Accordingly, in embodiments, predicted Bc data (e.g., time dependent Bc data) can be converted to GHB parameters, which GHB parameters can subsequently be utilized in hydraulics and the determination of circulation pressures.

It is noted that, in embodiments, tested Bc(t) data can be utilized in place of or in addition to the predicted Bc(t) from the consistency model (e.g., in a hydraulic parameter calculation). It is further noted that time dependent GHB parameters for use in hydraulics calculation(s) can, in embodiments, be calculated by other means alternatively or in addition to those described herein. That is, while various non-limiting techniques are demonstrated herein, any suitable method or technique may be used to obtain time dependent GHB parameters, as would be apparent to a person of ordinary skill in the art with the aid of the present disclosure.

Also provided herein is a system comprising: a processor (782 FIG. 8 described hereinbelow); a memory (786/788 of FIG. 8); and an analysis program stored in the memory, wherein the analysis program is configured, when executed on the processor, to: receive, during a cementing operation with a cement slurry 50, one or more adjusted parameters via a user interface (e.g., 790, FIG. 8); use the one or more adjusted parameters in a consistency model; and generate an updated consistency vs. time curve predicted by the consistency model, wherein the consistency model predicts the consistency of the cement slurry as a function of time (Bc(t)). As discussed hereinabove, the consistency model can predict Bc(t) via: a dissolution module [DM] that models the consistency behavior of the cement slurry 50 during dissolution of particles therein from the initial time (t₀) to a dissolution time (t_{d}) at an end of dissolution; a thermal thinning module [TTM] that models thermal thinning of the cement slurry 50; a static gelling module [SGM] that models consistency behavior of the cement slurry 50 during shut down from an initial shutdown time (t_{sd, start}) to an end of the shutdown (t_{sd, end}); and a hydration module [HM] that models the consistency behavior of the cement slurry 50 during hydration of cementitious particles therein from an initial hydration time (tₕₒ) to the thickening time (t_{TT}). As discussed hereinabove with reference to Equation (1), Bc(t)=Bc(i)[DM][TTM][SGM][HM], wherein Bc(i) is the initial consistency of the cement slurry at to.

In embodiments, the analysis program is further configured to: receive rheology data of the cement slurry at one or more temperatures (e.g., such as obtained at 7 of FIG. 1B); determine GHB parameters based on the rheology data (e.g., such at 8 of FIG. 1B); and calculate adjusted GHB parameters as a function of time (e.g., such as at 21 of FIG. 1).

In embodiments, the memory can further comprise a hydraulics program (e.g., such as iCem® Service), wherein the hydraulics program is configured to, when executed on the processor: utilize the adjusted GHB parameters to determine a hydraulic parameter of the cementing operation. Without limitation, the hydraulic parameter can comprise an equivalent circulation density (ECD), a surface pressure, or a combination thereof.

In embodiments, the dissolution module [DM] can be as described hereinabove (e.g., with reference to Equations 2A-2C, wherein the dissolution module [DM] is only active if t_{d}>0, wherein: if t_{d}=0, [DM]=1.0, when t<t_{d}, [DM]=e\hat{ }(k_{d} t/t_{d}), when t≥t_{d}, [DM]=[DM] at t=t_{d}, wherein to is the initial time (e.g., time zero) when the cement slurry is mixed, and t_{d} is the time at the end of dissolution. As depicted in FIG. 3B, the one or more adjusted parameters can comprise k_{d}, t_{d}, or a combination thereof. Other dissolution models not described herein can be deployed to model the dissolution of chemical additives and wetting of particle surfaces, in embodiments.

In embodiments, the thermal thinning module [TTM] is as described hereinabove (e.g., with reference to Eq. (3), wherein [TTM]=e\hat{ }(((Delta E)/R)((1/T)−(1/Tᵢ)), wherein Delta E is an activation energy for thermal thinning (e.g., typically ranging from 0 to 5,000 cal/gmol), R is the universal gas constant (e.g., 1.986 cal/gmol-K), Tᵢ is a temperature at the beginning of the TT test in Kelvin, and T is temperature at any given time t in Kelvin). As depicted in FIG. 4B, the one or more adjusted parameters comprises Delta E, Tᵢ, or a combination thereof. For some exotic cementing formulations, the value of Delta E may exceed 5,000 cal/gmol-K.

In embodiments, the static gelling module [SGM] is as described hereinabove (e.g., with reference to Eq. (4), wherein: [SGM] is only active if there is a shut down (e.g., Shut Down is True or 1 or 0), and when t≥t_{sd}, [SGM]=e\hat{ }((k_{sd}(t_{sd,end}−t_{sd,start}))), wherein t_{sd,start} is a time at which shut down starts, t_{sd,end} is a time at shut down ends, and k_{sd} is a coefficient of static gelling reaction. Accordingly, [SSM] can equal 1 if there is no shut down. As depicted in FIG. 5B, the one or more adjusted parameters can include an indication (e.g., TRUE or FALSE, YES or NO, 1 or ≠1) of whether or not there is a shut down, a start time of a shut down during the cementing operation (t_{sd,start/actual}), an end time of the shut down during the cementing operation (t_{sd,end/actual}), or a combination thereof.

In embodiments of the system, the hydration module [HM] is as described hereinabove (e.g., with reference to Eq. (4), wherein: [HM]=e\hat{ }((k_{hyd})(t−tₕ₀)^{α}) from t=tₕ₀ to t=t_{TT}, wherein t_{TT}=thickening time at 70Bc, tₕₒ is a time when hydration begins, t=time, α (alpha_hyd) is a pseudo reaction order coefficient, and k_{hyd} is a hydration coefficient. As depicted in FIG. 6B, the one or more adjusted parameters can comprise tₕ₀.

FIG. 8 illustrates a computer system 780 suitable for implementing one or more steps of or embodiments of the methods disclosed herein. The computer system 780 includes a processor 782 (which may be referred to as a central processor unit or CPU) that is in communication with memory devices including secondary storage 784, read only memory (ROM) 786, random access memory (RAM) 788, input/output (I/O) devices 790, and network connectivity devices 792. The processor 782 may be implemented as one or more CPU chips.

It is understood that by programming and/or loading executable instructions onto the computer system 780, at least one of the CPU 782, the RAM 788, and the ROM 786 are changed, transforming the computer system 780 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 780 is turned on or booted, the CPU 782 may execute a computer program or application. For example, the CPU 782 may execute software or firmware stored in the ROM 786 or stored in the RAM 788. In some cases, on boot and/or when the application is initiated, the CPU 782 may copy the application or portions of the application from the secondary storage 784 to the RAM 788 or to memory space within the CPU 782 itself, and the CPU 782 may then execute instructions of which the application is comprised. In some cases, the CPU 782 may copy the application or portions of the application from memory accessed via the network connectivity devices 792 or via the I/O devices 790 to the RAM 788 or to memory space within the CPU 782, and the CPU 782 may then execute instructions of which the application is comprised. During execution, an application may load instructions into the CPU 782, for example load some of the instructions of the application into a cache of the CPU 782. In some contexts, an application that is executed may be said to configure the CPU 782 to do something, e.g., to configure the CPU 782 to perform the function or functions promoted by the subject application. When the CPU 782 is configured in this way by the application, the CPU 782 becomes a specific purpose computer or a specific purpose machine.

The secondary storage 784 is typically comprised of one or more disk drives or tape drives and is used for non-volatile storage of data and as an over-flow data storage device if RAM 788 is not large enough to hold all working data. Secondary storage 784 may be used to store programs which are loaded into RAM 788 when such programs are selected for execution. The ROM 786 is used to store instructions and perhaps data which are read during program execution. ROM 786 is a non-volatile memory device which typically has a small memory capacity relative to the larger memory capacity of secondary storage 784. The RAM 788 is used to store volatile data and perhaps to store instructions. Access to both ROM 786 and RAM 788 is typically faster than to secondary storage 784. The secondary storage 784, the RAM 788, and/or the ROM 786 may be referred to in some contexts as computer readable storage media and/or non-transitory computer readable media.

I/O devices 790 may include printers, video monitors, electronic displays (e.g., liquid crystal displays (LCDs), plasma displays, organic light emitting diode displays (OLED), touch sensitive displays, etc.), keyboards, keypads, switches, dials, mice, track balls, voice recognizers, card readers, paper tape readers, or other well-known input devices.

The network connectivity devices 792 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 792 may enable the processor 782 to communicate with the Internet or one or more intranets. With such a network connection, it is contemplated that the processor 782 might receive information from the network, or might output information to the network (e.g., to an event database) in the course of performing the above-described method steps. Such information, which is often represented as a sequence of instructions to be executed using processor 782, may be received from and outputted to the network, for example, in the form of a computer data signal embodied in a carrier wave.

Such information, which may include data or instructions to be executed using processor 782 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several known methods. The baseband signal and/or signal embedded in the carrier wave may be referred to in some contexts as a transitory signal.

The processor 782 executes instructions, codes, computer programs, scripts which it accesses from hard disk, floppy disk, optical disk (these various disk based systems may all be considered secondary storage 784), flash drive, ROM 786, RAM 788, or the network connectivity devices 792. While only one processor 782 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 784, for example, hard drives, floppy disks, optical disks, and/or other device, the ROM 786, and/or the RAM 788 may be referred to in some contexts as non-transitory instructions and/or non-transitory information.

In embodiments, the computer system 780 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 780 to provide the functionality of a number of servers that is not directly bound to the number of computers in the computer system 780. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third party provider. Some cloud computing environments may comprise cloud computing resources owned and operated by the enterprise as well as cloud computing resources hired and/or leased from a third party provider.

In embodiments, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the computer system 780, at least portions of the contents of the computer program product to the secondary storage 784, to the ROM 786, to the RAM 788, and/or to other non-volatile memory and volatile memory of the computer system 780. The processor 782 may process the executable instructions and/or data structures in part by directly accessing the computer program product, for example by reading from a CD-ROM disk inserted into a disk drive peripheral of the computer system 780. Alternatively, the processor 782 may process the executable instructions and/or data structures by remotely accessing the computer program product, for example by downloading the executable instructions and/or data structures from a remote server through the network connectivity devices 792. The computer program product may comprise instructions that promote the loading and/or copying of data, data structures, files, and/or executable instructions to the secondary storage 784, to the ROM 786, to the RAM 788, and/or to other non-volatile memory and volatile memory of the computer system 780.

In some contexts, the secondary storage 784, the ROM 786, and the RAM 788 may be referred to as a non-transitory computer readable medium or a computer readable storage media. A dynamic RAM embodiment of the RAM 788, likewise, may be referred to as a non-transitory computer readable medium in that while the dynamic RAM receives electrical power and is operated in accordance with its design, for example during a period of time during which the computer system 780 is turned on and operational, the dynamic RAM stores information that is written to it. Similarly, the processor 782 may comprise an internal RAM, an internal ROM, a cache memory, and/or other internal non-transitory storage blocks, sections, or components that may be referred to in some contexts as non-transitory computer readable media or computer readable storage media.

The cement slurry 50 can comprise a cementitious material. The cementitious material can comprise calcium, aluminum, silicon, oxygen, iron, and/or sulfur, resins, latex, etc. The cementitious material can comprise Portland cement, pozzolana cement, gypsum cement, shale cement, cement kiln dust, acid/base cement, phosphate cement, high alumina content cement, slag cement, silica cement, high alkalinity cement, magnesia cement, hollow glass beads, or a combination thereof. In embodiments, “high alumina content cement” refers to a cement having an alumina concentration in the range of from about 40 wt. % to about 80 wt. % by a weight of the high alumina content cement. In embodiments, “high alkalinity cement” refers to a cement having a sodium oxide concentration in the range of from about 1.0 wt. % to about 2.0 wt. % by a weight of the high alkalinity cement.

In embodiments, the cement slurry does not comprise a Portland cement, is substantially free of a Portland cement, or comprises zero or less than 10, 9, 8, 7, 6, 5, 4, 3, 2, 1, 0.1, 0.01, 0.001, or 0.0001 weight percent Portland cement. In embodiments, the cement slurry can comprise a Portland cement. In embodiments, the cement slurry 50 is not a Portland cement. Portland cements that are suited for use in the disclosed cement slurry 50 include, but are not limited to, Class A, C, G, H, low sulfate resistant cements, medium sulfate resistant cements, high sulfate resistant cements, or combinations thereof. The class A, C, G, and H cements are classified according to API Specification 10. Additional examples of Portland cements suitable for use in the present disclose include, without limitation, those classified as ASTM Type I, II, III, IV, or V. In embodiments, the cementitious material comprises a Class G cement, a Class H cement, a Class A cement, a Class C cement, or combinations thereof. In embodiments, the cement slurry 50 comprises a non-API cement.

The cementitious material can be present in the cement slurry 50 in an amount of from about 0.01 wt. % to about 90 wt. % based on the total weight of the cement slurry 50, alternatively from about 0.01 wt. % to about 75 wt. %, alternatively from about 0.01 wt. % to about 50 wt. %, or alternatively from about 0.04 wt. % to about 25 wt. %.

The cement slurry 50 can include an aqueous fluid. Generally, the aqueous fluid can be from any source, provided that it does not contain an amount of components that may undesirably affect the other components in the cement slurry 50. For example, the aqueous fluid can comprise fresh water, surface water, ground water, produced water, salt water, sea water, brine (e.g., underground natural brine, formulated brine, etc.), or a combination thereof. In embodiments, the aqueous fluid comprises a brine. In embodiments, the brine includes monovalent or divalent salts such as, sodium chloride, sodium bromide, potassium bromide, potassium chloride, magnesium chloride, calcium chloride, calcium bromide, potassium formate, cesium formate, lithium chloride, lithium bromide, sodium formate, lithium formate, ammonium chloride, tetramethyl ammonium chloride, choline chloride, potassium acetate, or a combination thereof. A formulated brine can be produced by dissolving one or more soluble salts in water, a natural brine, or sea water. The brine can be saturated or unsaturated.

The aqueous fluid can be present in the cement slurry 50 in an amount effective to provide a pumpable slurry, such as a slurry having desired (e.g., job or service specific) rheological properties. In embodiments, the aqueous fluid can be present in the cement slurry 50 in an amount of from about 5 vol. % to about 99 vol. % based on the total volume of the cement slurry 50, alternatively from about 10 vol. % to about 99 vol. %, or alternatively from about 25 vol. % to about 95 vol. %.

Cement slurry 50 can further comprise one or more additional components. The one or more additional component(s) can be present in any suitable amount. For example, generally, the one or more additional components can be present in the cement slurry 50 in an amount of from about 0.01 wt. % to about 60 wt. % based on the total weight of the cement slurry 50, alternatively from about 0.01 wt. % to about 30 wt. %, alternatively from about 0.01 wt. % to about 15 wt. %, or alternatively from about 0.01 wt. % to about 5 wt. %.

In embodiments, the cement slurry 50 further comprises one or more additives. The one or more additives can comprise weighting agents, retarders, accelerators, activators, gas control additives, lightweight additives, gas-generating additives, mechanical-property-enhancing additives, lost-circulation materials, filtration-control additives, fluid-loss-control additives, defoaming agents, foaming agents, transition time modifiers, thixotropic additives, suspending agents, acid soluble materials, or combinations thereof. The oxidative breaker can include bromate, persulfate, perborate, and perbromate, for example. With the benefit of this disclosure, one of ordinary skill in the art should be able to recognize and select one or more suitable additives for use in the cement slurry. In embodiments, the one or more additives are present in the cement slurry 50 in an amount of from about 0.001 wt. % to about 75 wt. %, based on the total weight of the cement slurry 50, alternatively from about 0.1 wt. % to about 70 wt. %, or alternatively from about 1 wt. % to about 50 wt. %.

Rheology results of the cement slurry 50 can be measured. Viscosity of the cement slurry 50 can be converted from “rheology dial readings,” which herein refers to dial readings on a FANN© viscometer at different rotational speeds (e.g., 300 revolutions per minute (rpm) to 3 rpm), when measured in accordance with test standard API-RP-10B-2. For example, the FANN® viscometer is rotated at 300 rpm for 10 seconds and a value on the dial is read, the speed can then be changed to another rpm and a new value on the dial reading can be taken. There are a number of theoretical models known to those of ordinary skill in the art that can be used to convert the values from the dial readings at the different rpm's into viscosity (centipoises) and/or into the four GHB parameters.

In embodiments, at a speed of from about 3 rpm to about 300 rpm and atmospheric pressure, the cement slurry 50 has a rheology dial reading in a range of from about 1 to about 300, alternatively from about 3 to about 200, alternatively from about 5 to about 200, alternatively from about 5 to about 100, alternatively from about 5 to about 80, or alternatively from about 10 to about 50, when measured in accordance with test standard API-RP-10B-2. In embodiments, at a speed of about 3 rpm, and atmospheric pressure, the cement slurry 50 has a rheology dial reading in a range of from about 1 to about 50, alternatively from about 3 to about 40, alternatively from about 5 to about 40, or alternatively from about 10 to about 35, when measured in accordance with test standard API-RP-10B-2. In embodiments, the rheology dial readings described herein correspond to one or more temperatures in a range of from about 30° F. to about 500° F., alternatively in a range of from about 50° F. to about 400° F., alternatively in a range of from about 80° F. to about 250° F., alternatively in a range of from about 80° F. to about 200° F., or alternatively in a range of from about 80° F. to about 190° F. In embodiments, the rheology dial readings described herein correspond to all temperatures spanning a range of from about 30° F. to about 500° F., alternatively spanning a range of from about 50° F. to about 400° F., alternatively spanning a range of from about 80° F. to about 250° F., alternatively spanning a range of from about 80° F. to about 200° F., or alternatively spanning a range of from about 80° F. to about 190° F.

The cement slurry 50 can have a 10-second gel strength of from about 1 lb_{f}/100 ft² to about 100 lb_{f}/100 ft², alternatively from about 5 lb_{f}/100 ft² to about 85 lb_{f}/100 ft², alternatively from about 5 lb_{f}/100 ft² to about 75 lb_{f}/100 ft², alternatively from about 15 lb_{f}/100 ft² to about 75 lb_{f}/100 ft², or alternatively from about 10 lb_{f}/100 ft² to about 65 lb_{f}/100 ft², when measured in accordance with test standard API-RP-10B-2. The cement slurry 50 can exhibit a 10-second gel strength of from about 1 lb_{f}/100 ft² to about 100 lb_{f}/100 ft², from about 5 lb_{f}/100 ft² to about 75 lb_{f}/100 ft², or from about 10 lb_{f}/100 ft² to about 65 lb_{f}/100 ft², when measured in accordance with test standard API-RP-10B-2.

The cement slurry 50 can have a 10-minute gel strength of from about 5 lb_{f}/100 ft² to about 300 lb_{f}/100 ft², alternatively from about 30 lb_{f}/100 ft² to about 250 lb_{f}/100 ft², alternatively from about 50 lb_{f}/100 ft² to about 220 lb_{f}/100 ft², alternatively from about 5 lb_{f}/100 ft² to about 220 lb_{f}/100 ft², or alternatively from about 100 lb_{f}/100 ft² to about 200 lb_{f}/100 ft², when measured in accordance with test standard API-RP-10B-2. The cement slurry 50 can have a 10-minute gel strength in a range of from about 5 lb_{f}/100 ft² to about 300 lb_{f}/100 ft², when measured in accordance with test standard API-RP-10B-2.

In embodiments, the cement slurry 50 has a thickening time. The thickening time herein refers to the time required for the cement slurry 50 to achieve 70. Bearden units of Consistency (Bc) after preparation of the cement slurry 50. At about 70 Bc, the cement slurry 50 undergoes a conversion from a pumpable fluid state to a non-pumpable gel. In order to keep the cement slurry 50 in a pumpable state for an appropriate amount of time, additives such as retarders, friction reducers, and accelerators can be added to modulate the pump time by shortening or extending the thickening time. A measurement of Bc can be considered a thickening time test which is performed on a moving fluid. As noted hereinabove, in a thickening time test, an apparatus including a pressurized consistometer can apply temperature and pressure to a slurry (e.g., the cement slurry 50) while the slurry is being stirred by a paddle. A resistor arm and potentiometer coupled to the paddle can provide an output of torque that is in units of Bc. Thickening time can be measured in accordance with test standard API-RP-10B-2.

In embodiments, at about 3,000 psi, the cement slurry 50 has a thickening time to achieve about 70 Bc ranging from about 1 hours to about 15 hours, alternatively from about 2 hours to about 12 hours, alternatively from about 3 hours to about 12 hours, alternatively from about 4 hours to about 12 hours, or alternatively from about 5 hours to about 12 hours, when measured in accordance with test standard API-RP-10B-2.

Compressive strength is generally the capacity of a material or structure to withstand axially directed compression forces. The compressive strength of a composition can be measured at a specified time (e.g., 24 hours) after a cement blend has been mixed with water and the resultant cement slurry is maintained under specified temperature and pressure conditions to form a hardened, set cement. For example, compressive strength can be measured at a time in the range of from about 12 to about 72 hours (or longer) after the cement slurry is mixed, and the cement slurry is maintained typically at a temperature of from 0° C./32° F. to about 204° C./400° F. and a suitable pressure, during which time the cement slurry can set into a hardened mass. Compressive strength can be measured by either a destructive method or non-destructive method. The destructive method physically tests the strength of hardened samples at various points in time by crushing the samples in a compression-testing machine. The compressive strength is calculated from the failure load divided by the cross-sectional area resisting the load and is reported in units of pound-force per square inch (psi). Non-destructive methods can employ an ultrasonic cement analyzer (UCA). A UCA can be available from FANN® Instrument Company, Houston, TX. Compressive strengths can be determined in accordance with API RP 10B-2, Recommended Practice for Testing Well Cements, First Edition, July 2005. Sonic strength is a measure of compressive strength based on “transit time” of soundwaves through the cement. The sonic strength is an indicator of crush compressive strength and is often used to monitor compressive strength versus time.

The cement slurry 50 can have a compressive strength evaluated by noting the time for the cement slurry 50 to reach 100 psi compressive strength (also referred to as “time to reach 100 psi”) as measured in an ultrasonic cement analyzer (UCA) test in accordance with test standard API-RP-10B-2. The time to reach 100 psi under static conditions in a UCA can be used as an estimation of the wait on cement time of a composition. At about 3,000 psi, the cement slurry 50 can have a time to reach 100 psi of from about 1 hours to about 25 hours, alternatively from about 2 hours to about 22 hours, alternatively from about 3 hours to about 22 hours, alternatively from about 4 hours to about 20 hours, alternatively from about 4 hours to about 16 hours, alternatively from about 4 hours to about 12 hours, or alternatively from about 5 hours to about 12 hours, when measured in a UCA in accordance with test standard API-RP-10B-2. In embodiments, the cement slurry 50 has a 24-hour compressive strength (also referred to as “24-hour crush strength” or “24-hour crush compressive strength”) measured in accordance with test standard API-RP-10B-2. The time is 24-hour period after preparation of the cement slurry 50. At about 3,000 psi, the 24-hour compressive strength can be in a range of from about 50 psi to about 8,000 psi, alternatively from about 50 psi to about 6,000 psi, alternatively from about 100 psi to about 3000 psi, alternatively from about 200 psi to about 3000 psi, or alternatively from about 300 psi to about 2800 psi, when measured in a UCA in accordance with test standard API-RP-10B-2. In embodiments, the time to reach 100 psi compressive strength and/or the 24-hour compressive strength described herein correspond to one or more temperatures in a range of from about 30° F. to about 500° F., alternatively in a range of from about 50° F. to about 400° F., alternatively in a range of from about 80° F. to about 250° F., alternatively in a range of from about 80° F. to about 200° F., or alternatively in a range of from about 80° F. to about 190° F. In embodiments, the time to reach 100 psi compressive strength and/or the 24-hour compressive strength described herein correspond to all temperatures spanning a range of from about 30° F. to about 500° F., alternatively spanning a range of from about 50° F. to about 400° F., alternatively spanning a range of from about 80° F. to about 250° F., alternatively spanning a range of from about 80° F. to about 200° F., or alternatively spanning a range of from about 80° F. to about 190° F.

Plastic viscosity is the viscosity when extrapolated to infinite shear rate, e.g., the slope of the shear stress/shear rate line above yield point. The yield point refers to the resistance of a fluid to initial flow, or represents the stress required to start fluid movement. The cement slurry 50 of this disclosure can have any suitable plastic viscosity and yield point. At pressures ranging from about atmospheric to about 40,000 psi, the cement slurry 50 can have a plastic viscosity of from about 5 cP to about 1000 cP, alternatively from about 20 cP to about 900 cP, or alternatively from about 20 cP to about 800 cP. At pressures ranging from about atmospheric to about 40,000 psi, the cement slurry 50 can have a yield point of from about 1 lb_{f}/100 ft² to about 100 lb_{f}/100 ft², alternatively from about 2 lb_{f}/100 ft² to about 90 lb_{f}/100 ft², alternatively from about 3 lb_{f}/100 ft² to about 80 lb_{f}/100 ft², or alternatively from about 5 lb_{f}/100 ft² to about 70 lb_{f}/100 ft². The plastic viscosity and yield point can be calculated using Bingham Plastic model. The Bingham Plastic viscosity model is a special case of a GHB viscosity model wherein m and n=1.0. In embodiments, the plastic viscosity and/or the yield point described herein correspond to one or more temperatures in a range of from about 30° F. to about 500° F., alternatively in a range of from about 50° F. to about 400° F., alternatively in a range of from about 80° F. to about 250° F., alternatively in a range of from about 80° F. to about 200° F., or alternatively in a range of from about 80° F. to about 190° F. In embodiments, the plastic viscosity and/or yield point described herein correspond to all temperatures spanning a range of from about 30° F. to about 500° F., alternatively spanning a range of from about 50° F. to about 400° F., alternatively spanning a range of from about 80° F. to about 250° F., alternatively spanning a range of from about 80° F. to about 200° F., or alternatively spanning a range of from about 80° F. to about 190° F.

For a given composition density and/or operating temperature (e.g., a range of bottomhole circulating temperatures associated with a given wellbore service), concentration of the various components of the cement slurry 50 can be varied within the ranges disclosed herein to provide one or more of the parameters selected from (i) a rheology dial reading meeting the values disclosed herein, (ii) a 10-second gel strength meeting the values disclosed herein, (iii) a 10-minute gel strength meeting the values disclosed herein, (iv) a thickening time meeting the values disclosed herein, (v) a 100 psi compressive strength meeting the values disclosed herein, (vi) a 24-hour compressive strength meeting the values disclosed herein, (vii) a plastic viscosity meeting the values disclosed herein, (viii) a yield point meeting the values disclosed herein, or (ix) any combination of (i) to (viii).

The cement slurry 50 disclosed herein can have any suitable density. In embodiments, the density of the cement slurry 50 ranges from about 6 pounds per gallon (lb/gal) to about 25 lb/gal, alternatively from about 8 lb/gal to about 23 lb/gal, alternatively from about 9 lb/gal to about 22 lb/gal, or alternatively from about 10 lb/gal to about 22 lb/gal.

A cement slurry 50 can be prepared using any suitable method, such as batch mixing or continuous mixing. In one or more embodiments, the method comprises mixing components (e.g., the cementitious material, the aqueous fluid, additives, etc.) of the cement slurry 50 using mixing equipment (e.g., a jet mixer, re-circulating mixer, a batch mixer, a blender, a mixing head of a solid feeding system) to form a pumpable fluid. For example, all components of the cement slurry 50 can be added to a batch mixer and agitated until the desired amount of mixing is achieved. Alternatively, the cement slurry 50 can be added to a continuous mixer where components are metered into the mixer and a product of the cement slurry 50 is continuously withdrawn. Metering components can comprise, but are not limited to, pneumatic conveyance of dry solids, liquid additives, screw conveyance of particles and powders and etc.

After preparation, if the cement slurry 50 can be transported to and/or stored at the wellsite, the transportation and/or storage vessel can have an agitator, rotor, mixer, or the like to impart sufficient shear to the cement slurry 50 to maintain a flowable, pumpable composition (e.g., a slurry).

In embodiments, the cement slurry 50 is used for servicing a wellbore 522 penetrating a subterranean formation 520 (FIG. 10, discussed in more detail hereinbelow). The wellbore can have a Bottomhole Circulating Temperature (BHCT) of from about 30° F. to about 600° F., alternatively from about 50° F. to about 500° F., alternatively from about 50° F. to about 450° F., alternatively from about 50° F. to about 400° F., alternatively from about 80° F. to about 250° F., alternatively from about 50° F. to about 200° F., alternatively from about 80° F. to about 200° F., or alternatively from about 80° F. to about 190° F. In embodiments, the wellbore has a Bottomhole Static Temperature (BHST) of from about 30° F. to about 600° F., alternatively from about 50° F. to about 500° F., alternatively from about 50° F. to about 450° F., alternatively from about 50° F. to about 400° F., alternatively from about 80° F. to about 250° F., alternatively from about 50° F. to about 200° F., alternatively from about 80° F. to about 200° F., or alternatively from about 80° F. to about 190° F.

Also provided herein is a method of servicing a wellbore utilizing the cement slurry 50 and utilizing the consistency model of this disclosure to predict the consistency of the slurry 50 as a function of time (e.g., Bc(t) of the cement slurry 50) during the cementing operation (e.g., to predict t_{TT} when Bc reaches 70 Bc). With reference to FIG. 10, a method I of servicing a wellbore penetrating a subterranean formation according to this disclosure can comprise: introducing cement slurry 50 downhole (e.g., into an annulus 532, proximate a location of a lost circulation zone 520′, etc.), wherein, as described hereinabove, the cement slurry 50 comprises a cementitious material and an aqueous fluid (e.g., water); and allowing the cement slurry 50 to set (e.g., to provide a hardened cement).

The cement slurry 50 introduced downhole can have a thickening time to about 70 Bearden units of Consistency (Bc) in a range of from about 1 hours to about 15 hours at about 3,000 psi, when measured in accordance with test standard API-RP-10B-2. The consistency model is utilized during the cementing operation to predict the time until 70 Bearden units of Consistency (Bc), utilizing adjusted parameters as described herein.

Introducing the cement slurry 50 downhole can comprise pumping the cement slurry 50 into the wellbore 522 (FIG. 10, described hereinbelow). The cement slurry 50 can be pumped into the wellbore 522 via a drill pipe, coiled tubing, and/or a drill bit, in embodiments. The method can further comprise ceasing of the introducing the cement slurry 50 downhole before allowing the cement slurry 50 to set.

In embodiments, the method is utilized in a lost circulation operation, and introducing a cement slurry 50 downhole comprises introducing a thixotropic cement slurry 50 downhole proximate a location of a lost circulation zone 520′ of the subterranean formation 520. In such embodiments, the set cement 50 can block at least a portion of the lost circulation zone 520′. The set cement 50 can thus be utilized to reduce lost circulation by reducing or preventing flow of a drilling fluid from the wellbore 522 through the lost circulation zone 520′ and into the adjacent subterranean formation 520. The method can include allowing the thixotropic cement slurry 50 to flow into at least a portion of a lost circulation zone 520′.

An example cementing technique using a cement slurry 50 of this disclosure will now be described with reference to FIG. 9, which is a schematic of surface equipment 400 that can be utilized in the placement of a cement slurry 50, according to embodiments disclosure, and FIG. 5, which is a schematic of the placement of a cementitious composition into a subterranean formation 520 in accordance with embodiments of the disclosure. FIG. 4 illustrates surface equipment 400 that can be used in the placement of a cement slurry 50 in accordance with certain examples. It will be noted that while FIG. 4 generally depicts a land-based operation, those skilled in the art will readily recognize that the principles described herein are equally applicable to subsea operations that employ floating or sea-based platforms and rigs, without departing from the scope of the disclosure. As illustrated in FIG. 4, the surface equipment 400 can include a cementing unit 405, which can include one or more cement trucks. The cementing unit 405 can include mixing equipment 410 and pumping equipment 415. Control equipment 401 comprising computer(s) 780 can be configured to control the cementing operation (e.g., operation of the mixing equipment 410, pumping equipment 415, etc.).

Cementing unit 405, or multiple cementing units 405, can pump a cement slurry 50 of the type disclosed herein through a feed pipe 420 and to a cementing head 425 which conveys the cement slurry 50 downhole. Cement slurry 50 can displace other fluids present in the wellbore, such as drilling fluids and spacer fluids, which can exit the wellbore through an annulus (532, FIG. 10) and flow through pipe 435 to mud pit 440.

Referring to FIG. 10, the cement slurry 50 can be placed into a subterranean formation 520 in accordance with example embodiments. As illustrated, a wellbore 522 can be drilled into the subterranean formation 520. While wellbore 522 is shown extending generally vertically into the subterranean formation 520, the principles described herein are also applicable to wellbores that extend at an angle through the subterranean formation 520, such as horizontal and slanted wellbores. As illustrated, the wellbore 522 comprises walls 524 of the wellbore 522. In the illustrated embodiment, a surface casing 526 has been inserted into the wellbore 522. The surface casing 526 can be cemented to the walls 524 of the wellbore 522 by cement sheath 528. In the illustrated embodiment, one or more additional conduits (e.g., intermediate casing, production casing, liners, etc.), shown here as casing 530 can also be disposed in the wellbore 522. As illustrated, there is a wellbore annulus (i.e., annular space) 532 formed between the casing 530 and the walls 524 of the wellbore 522 and/or the surface casing 526. One or more centralizers 534 can be attached to the casing 530, for example, to centralize the casing 530 in the wellbore 522 prior to and during the cementing operation.

With continued reference to FIG. 10, the cement slurry 50 can be placed (e.g., pumped) down the interior of the casing 530. The cement slurry 50 can be allowed to flow down the interior of the casing 530 through the casing shoe 542 at the bottom of the casing 530 and up around the casing 530 into the wellbore annulus 532. The cement slurry 50 can be allowed to set in the wellbore annulus 532, for example, to form a cement sheath that supports and positions the casing 530 in the wellbore 522. Other techniques can also be utilized for introduction of the cement slurry 50. By way of example, reverse circulation techniques can be used that includes introducing the cement slurry 50 into the subterranean formation 520 by way of the wellbore annulus 532 instead of through the casing 530. In such embodiments, the method can comprise circulating the cement slurry 50 down through the wellbore annulus 532 and back up through the interior of the casing 530.

In embodiments, the cement slurry 50 displaces other fluids 536, such as drilling fluids and/or spacer fluids that can be present in the interior of the casing 530 and/or the wellbore annulus 532. At least a portion of the displaced fluids 536 can exit the wellbore annulus 532 via a flow line and be deposited, for example, in one or more retention pits (e.g., a mud pit 440 in FIG. 9). A bottom plug 544 can be introduced into the wellbore 522 ahead of the cement slurry 50, for example, to separate the cement slurry 50 from the fluids 536 that can be inside the casing 530 prior to cementing. After the bottom plug 544 reaches the landing collar 546, a diaphragm or other suitable device can rupture to allow the cement slurry 50 through the bottom plug 544. In FIG. 10, the bottom plug 544 is shown on the landing collar 546. In the illustrated embodiment, a top plug 548 can be introduced into the wellbore 522 behind the cement slurry 50. The top plug 548 can separate the cement slurry 50 from a displacement fluid 550 and also push the cement slurry 50 through the bottom plug 544.

In embodiments, the method disclosed herein further comprises circulating the cement slurry 50 down through a conduit (e.g., casing) and back up through an annular space (also referred to as an annulus or a wellbore annulus) between an outside wall of the conduit and a wall of the wellbore. In some other embodiments, the method disclosed herein further comprises circulating the cement slurry 50 down through an annular space between an outside wall of a conduit and a wall of the wellbore and back up through the conduit. The method can further comprise allowing at least a portion of the cement slurry 50 to set.

A method of servicing a wellbore 522 penetrating a subterranean formation 520 can comprise placing a cement slurry 50 of the type disclosed herein into the wellbore 522, and allowing at least a portion of the cement slurry 50 to set. The wellbore can have a conduit 530 (e.g., casing, production tubing, tubular, or other mechanical conveyance, etc.) disposed therein to form an annular space 532 between a wellbore wall 524 and an outer surface of the conduit. In embodiments, the method comprises placing a cement slurry 50 of the type disclosed herein into at least a portion of the annular space 532, and allowing at least a portion of the cement slurry 50 to set. The consistency model of Eq. (1) is utilized to monitor the cementing operation during the cementing operation.

In embodiments of the method disclosed herein, placing cement slurry 50 into at least a portion of the annular space 532 can be in different directions. In some embodiments, placing the cement slurry 50 comprises circulating the cement slurry 50 down through the conduit 520 and back up through the annular space 532. In embodiments, placing the cement slurry 50 comprises circulating the cement slurry 50 down through the annular space 532 and back up through the conduit 530. In embodiments, the conduit 530 comprises casing.

In embodiments, a method of this disclosure can comprise introducing a cement slurry 50 proximate the location of a lost circulation zone 520′ in the wellbore 522. As previously mentioned, lost circulation zones are often encountered in a wellbore 522. A lost circulation zone 520′ can comprise a depleted zone, a zone of relatively low pressure, a zone having naturally-occurring fractures, a weak zone having fracture gradients exceeded by the hydrostatic pressure of the drilling fluid, or combinations thereof. The lost circulation zone can be in an uncased portion of the wellbore 522, such as a zone having naturally-occurring fractures and/or fractures induced during the drilling operation. The lost circulation zone 520′ can comprise flow paths between the wellbore 522 and the subterranean formation 520, where fluids can flow from the wellbore 522 to the subterranean formation 520 or in a reverse direction.

Service provided by a wellbore servicing fluid in the lost circulation zone 520′ can be more difficult to achieve. In one scenario, a drilling fluid may be lost to the formation, resulting in the circulation of the drilling fluid in the wellbore being too low to allow for further drilling of the wellbore. The cement slurry 50 can be used to seal the lost circulation zones 520′ to prevent the uncontrolled flow of fluids into or out of the lost circulation zones 520′, e.g., lost drilling fluid circulation, crossflows, underground blow-outs and the like.

In applications, a secondary cement/sealant composition may be lost to the formation as it is being placed in the wellbore, thereby rendering the secondary operation ineffective in maintaining isolation of the formation 520. In addition to drilling fluids, embodiments of the present disclosure may also be used to control lost circulation problems encountered with other fluids, for example, spacer fluids, completion fluids (e.g., completion brines), fracturing fluids, and cement compositions that may be placed into a wellbore 522.

In embodiments, a method of servicing a wellbore penetrating a subterranean formation comprises: drilling the wellbore 522 with a drill bit connected to a drill pipe, determining a location of a lost circulation zone 520′ in the wellbore 522, and introducing a cement slurry 50 of the type disclosed herein proximate the location of the lost circulation zone 520′. Drilling the wellbore 522 can comprise circulating a drilling fluid via the drill pipe. In embodiments, the lost circulation zone 520′ is in an uncased portion of the wellbore 522.

In embodiments, introducing the cement slurry 50 comprises pumping the cement slurry 50 into the wellbore. The cement slurry 50 can be pumped through one or more openings at the end of the string of the drill pipe. In embodiments, the cement slurry 50 can be pumped through the drill pipe and the drill bit.

The method can further comprise allowing the cement slurry 50 to flow into at least a portion of the lost circulation zone 520′. Once placed (e.g., into the lost circulation zone 520′), the cement slurry 50 can be allowed to set, thus a hardened mass (i.e., to provide a set or hardened cement) can be formed. In embodiments, as noted hereinabove, the set cement blocks fluid flow (e.g., inflow or outflow). For example, in embodiments, the set cement can reduce lost circulation by reducing or preventing flow of the drilling fluid from the wellbore 522 through the lost circulation zone 520′ and into the adjacent subterranean formation 520, which allows for continued drilling. In embodiments, the method further comprises ceasing introducing the cement slurry 50 before allowing the cement slurry 50 to set. Upon cessation of introducing (e.g., pumping) the cement slurry 50, the cement slurry 50 can exhibit increasing gel strength to set.

In embodiments, the drilling is discontinued prior to introducing the cement slurry 50 in a desired location. In such embodiments, the method can further comprise resuming drilling of the wellbore 522 after allowing the cement slurry 50 to set.

Although primary and lost circulation cementing operations are discussed herein, the cement slurry 50 can be utilized in any cementing operation according to this disclosure, provided the cementing operation is monitored as described herein via the use of the consistency model to predict the thickening time to 70 Bc. The cement slurry 50 can be positioned at any desired location downhole (e.g., annular space 532 between wellbore and conduit, to form a plug in a flowpath into or through the wellbore (e.g., to plug a well, plug a loss or influx zone, plug a perforated interval, etc.).

Various benefits may be realized by utilization of the presently disclosed methods and compositions. Conventional simulators assume that a cement slurry exhibits time-independent behavior. The herein disclosed system and method can be utilized to improve the accuracy of simulation results by including the effect of cement slurry consistency toward viscosity and friction pressure. The method comprises modeling the physico-chemical phenomena (e.g., dissolution, thermal thinning, static gelling, and hydration) that are taking place in cementing.

The herein disclosed method enables a more precise estimation of how cement slurry rheology will change over time as the slurry undergoes dissolution, thermal thinning, shutdown, and cement thickening, thereby improving the reliability of hydraulic calculations and ensuring the safety and success of critical operations. In addition, engineers can gain valuable insights into how frictional pressure would change if the pump schedule on a cementing job were to deviate from the original plan. The herein disclosed modeling method provides a fundamental building blocks that can support the development of the first “Fully Automated Cement Job.”

For a given well configuration, when effect of consistency is neglected, friction pressure can be underestimated by as much as 129.8%. Conventional studies examining the effect of slurry consistency on frictional pressure have been performed by repeatedly collecting samples from thickening time tests and measuring viscosity at time intervals until a limit of the viscometer is exceeded. Performing cement rheology tests multiple times alongside thickening time tests allows measurement of cement rheology beyond API standard 30 minute conditioning time. When the rheological parameters are adjusted at different time intervals, the resulting model leads to significantly higher predicted frictional pressures compared to scenarios where such adjustments are not made. However, such test procedures are cumbersome and require large amount of testing to be applicable to other design cases. Unlike such conventional methods, the herein disclosed method does not require any additional/repeat testing, and is applicable to a wide range of cement slurries.

### Additional Disclosure

The following is provided as additional disclosure for combinations of features and embodiments of the present disclosure.

In a first embodiment, a method comprises: providing a consistency model that predicts consistency of a cement slurry design as a function of time (Bc(t)); performing a cementing operation with a cement slurry having a same composition as that of the cement slurry design; and utilizing the consistency model to predict the Bc(t) of the cement slurry during the cementing operation.

A second embodiment can include the method of the first embodiment, wherein providing the consistency model comprises: obtaining consistency versus time (Bc(t)) data for a sample of the cement slurry design from an initial time (to) of mixing sample of the cement slurry design to a thickening time (t_{TT}) at which the consistency of the sample reaches 70 Bc (Beardon consistency units); and utilizing the consistency versus time data to determine coefficients utilized by the consistency model to predict the consistency of the cement slurry design as a function of time, wherein utilizing the consistency versus time data to determine the coefficients comprises statistical regression until a predicted Bc(t) simulates/models the Bc(t) data within a specified margin of error.

A third embodiment can include the method of the second embodiment, wherein the consistency model predicts the consistency as a function of time (Bc(t)) via: a dissolution module [DM] that models the consistency behavior during dissolution of particles therein from the initial time (to) to a dissolution time (t_{d}) at an end of dissolution; a thermal thinning module [TTM] that models thermal thinning, if present, during initial ramping of the temperature; a static gelling module [SGM] that models consistency behavior of the cement slurry during a shut down, if present, from an initial shutdown time (t_{sd, start}) to an end of the shutdown (t_{sd, end}); and/or a hydration module [HM] that models the consistency behavior during hydration of cementitious particles from an initial hydration time (tₕₒ) to the thickening time (t_{TT}), wherein Bc(t)=Bc(i) [DM][TTM][SGM][HM], wherein Bc(i) is the initial consistency at to.

In a fourth embodiment, a system comprises: a processor; a memory; and an analysis program stored in the memory, wherein the analysis program is configured, when executed on the processor, to: receive, during a cementing operation with a cement slurry, one or more adjusted parameters via a user interface; use the one or more adjusted parameters in a consistency model; and generate an updated consistency vs. time curve predicted by the consistency model, wherein the consistency model predicts the consistency of the cement slurry as a function of time (Bc(t)) via: a dissolution module [DM] that models the consistency behavior of the cement slurry during dissolution of particles therein from the initial time (to) to a dissolution time (t_{d}) at an end of dissolution; a thermal thinning module [TTM] that models thermal thinning of the cement slurry; a static gelling module [SGM] that models consistency behavior of the cement slurry during shut down from an initial shutdown time (t_{sd, start}) to an end of the shutdown (t_{sd, end}); and a hydration module [HM] that models the consistency behavior of the cement slurry during hydration of cementitious particles therein from an initial hydration time (tₕₒ) to the thickening time (t_{TT}), wherein Bc(t)=Bc(i)[DM][TTM][SGM][HM], wherein Bc(i) is the initial consistency of the cement slurry at t₀.

A fifth embodiment can include the system of the fourth embodiment, wherein the analysis program is further configured to: receive rheology data of the cement slurry at one or more temperatures; determine GHB parameters based on the rheology data; and calculate adjusted GHB parameters as a function of time.

A sixth embodiment can include the system of the fifth embodiment, wherein the memory further comprises a hydraulics program, wherein the hydraulics program is configured to, when executed on the processor: utilize the adjusted GHB parameters to determine a hydraulic parameter of the cementing operation.

A seventh embodiment can include the system of the sixth embodiment, wherein the hydraulic parameter comprises an equivalent circulation density (ECD), a surface pressure, or a combination thereof.

An eighth embodiment can include the system of any one of the fourth to seventh embodiments, wherein the dissolution module [DM] is only active if t_{d}>0, wherein: if t_{d}=0, [DM]=1.0, when t<t_{d}, [DM]=e\hat{ }(k_{d} t/t_{d}), when t≥t_{d}, [DM]=[DM] at t=t_{d}, wherein to is the initial time (e.g., time zero) when the cement slurry is mixed, and t_{d} is the time at the end of dissolution. Bcᵢ is an initial Bc reading at t₀, and Bc_{td} is a maximum Bc reading at the end of dissolution (e.g., at t_{d}).

A ninth embodiment can include the system of the eighth embodiment, wherein the one or more adjusted parameters comprises k_{d}, t_{d}, Bcᵢ, Bc_{td}, or a combination thereof.

A tenth embodiment can include the system of any one of the fourth to ninth embodiments, wherein: [TTM]=e\hat{ }(((Delta E)/R)((1/T)−(1/Tᵢ)), wherein Delta E is an activation energy for thermal thinning (e.g., typically ranging from 0 to 5,000 cal/gmol), R is the universal gas constant (e.g., 1.986 cal/gmol-K), Tᵢ is a temperature at the beginning of the TT test in Kelvin, and T is temperature at any given time t in Kelvin.

An eleventh embodiment can include the system of the tenth embodiment, wherein the one or more adjusted parameters comprises Delta E, Tᵢ, or a combination thereof.

A twelfth embodiment can include the system of any one of the fourth to tenth embodiments, wherein: [SGM] is only active if there is a shut down (e.g., Shut Down is True), and when t≥t_{sd}, [SGM]=e\hat{ }((k_{sd}(t_{sd,end}−t_{sd,start}))), wherein t_{sd,start} is a time at which shut down starts, t_{sd,end} is a time at shut down ends, and k_{sd} is a coefficient of static gelling reaction.

A thirteenth embodiment can include the system of the twelfth embodiment, wherein the one or more adjusted parameters comprises an indication (e.g., TRUE or FALSE, YES or NO, 1 or ≠1) of whether or not there is a shut down, a start time of a shut down during the cementing operation (t_{sd,start/actual}), an end time of the shut down during the cementing operation (t_{sd,end/actual}), or a combination thereof.

A fourteenth embodiment can include the system of any one of the fourth to thirteenth embodiments, wherein: [HM]=e\hat{ }((k_{hyd})(t−tₕ₀)^{α}) from t=tₕ₀ to t=t_{TT}, wherein t_{TT}=thickening time at 70Bc, tₕₒ is a time when hydration begins, t=time, α (alpha_hyd) is a pseudo reaction order coefficient, and k_{hyd} is a hydration coefficient. Bc_th_o Bcₕ₀ is the Beardon consistency at the time tₕ₀ when hydration begins.

A fifteenth embodiment can include the system of the fourteenth embodiment, wherein the one or more adjusted parameters comprise tₕ₀, BCₕ₀, or a combination thereof.

In a sixteenth embodiment, a method comprises: obtaining consistency versus time (Bc(t)) data from a thickening time (TT) test for a cement slurry design from an initial time (to) of mixing the cement slurry design to a thickening time (t_{TT}) at which the consistency reaches 70 Bc (Beardon consistency units); and utilizing the consistency versus time data to determine parameters for a consistency model to predict the consistency of the cement slurry design as a function of time.

A seventeenth embodiment can include the method of the sixteenth embodiment, wherein the consistency model predicts the consistency of the cement slurry design as a function of time (Bc(t)) via: a dissolution module [DM] that models the consistency behavior of the cement slurry design during dissolution of particles therein from the initial time (to) to a dissolution time (t_{d}) at an end of dissolution; a thermal thinning module [TTM] that models thermal thinning, if present, of the cement slurry design from during initial ramping of the temperature during the obtaining of the consistency versus time (Bc(t)) data for the cement slurry design; a static gelling module [SGM] that models consistency behavior of the cement slurry design during a shut down from a start of the shutdown (t_{sd, start}) to an end of the shutdown (t_{sd, end}); and a hydration module [HM] that models the consistency behavior of the cement slurry design during hydration of cementitious particles therein from an initial hydration time (tₕₒ) to the thickening time (t_{TT}) at which the consistency reaches 70 Bc, wherein Bc(t)=Bc(i)[DM][TTM][SGM][HM], wherein Bc(i) is the initial consistency of the cement slurry deign at to.

An eighteenth embodiment can include the method of the seventeenth embodiment, wherein the dissolution module [DM] is only active if t_{d}>0, wherein: if t_{d}=0, [DM]=1.0, when t<t_{d}, [DM]=e\hat{ }(k_{d} t/t_{d}), when t≥t_{d}, [DM]=[DM] at t=t_{d}, wherein t₀ is the initial time of mixing, at the beginning of the TT test, Bcᵢ is the initial consistency (e.g., Bc reading) at the beginning of the TT test, and t_{d} is the dissolution time at an end of dissolution (e.g., when a maximum Bc reading, Bc_{td}, is obtained).

A nineteenth embodiment can include the method of the seventeenth or eighteenth embodiment, wherein: [TTM]=e\hat{ }(((Delta E)/R)((1/T)−(1/Tᵢ)), wherein Delta E is an activation energy for thermal thinning (e.g., typically ranging from 0 to 5,000 cal/gmol), R is the universal gas constant (e.g., 1.986 cal/gmol-K), Tᵢ is a temperature at the beginning of the TT test in Kelvin, and T is temperature at any given time t in Kelvin.

A twentieth embodiment can include the method of any one of the seventeenth to nineteenth embodiments, wherein: [SGM] module is only active if there is a shut down (e.g., Shut Down is True), and when t≥t_{sd}, [SGM]=e\hat{ }((k_{sd}(t_{sd,end}−t_{sd,start}))), wherein t_{sd,start} is a time at which rotation speed is stopped at the start of shut down, t_{sd,end} is a time at which rotation speed is resumed (e.g., to 150 RPM) after the shut down, and k_{sd} is a coefficient of static gelling reaction.

A twenty first embodiment can include the method of any one of the seventeenth to twentieth embodiments, wherein: [HM]=e\hat{ }((k_{hyd})(t−tₕ₀)^{α}) from t=tₕ₀ to t=t_{TT}, wherein t_{TT}=thickening time at 70Bc, tₕₒ is a time when hydration begins, t=time, α (e.g., alpha_hyd in FIG. 6B) is a pseudo reaction order coefficient, and k_{hyd} is a hydration coefficient. Bc_th_o Bc(t=ho) is the Beardon consistency at the time tₕ₀ when hydration begins.

A twenty second embodiment can include the method of any one of the sixteenth to twenty first embodiments, wherein utilizing the consistency versus time data to determine parameters for the consistency model to predict the consistency of the cement slurry design as the function of time comprises utilizing statistical regression to determine the parameters for the consistency model.

A twenty third embodiment can include the method of any one of the sixteenth to twenty second embodiments further comprising: performing the TT test to obtain the consistency versus time (Bc(t)) data for the cement slurry from the initial time (t₀) of mixing the cement slurry design to the thickening time (t_{TT}) at which the consistency reaches 70 Bc (Beardon consistency units).

A twenty fourth embodiment can include the method of the twenty third embodiment further comprising performing the thickening time (TT) test by: loading a sample of the cement slurry design into a cup (e.g., either a “conventional HPHT cup” or a “thixotropic cup”); starting rotation of the cup at a constant speed (e.g., of 150 RPM) at time to; ramping the temperature from an initial temperature Tᵢ to a second temperature (e.g., BHCT) and a selected pressure in a temperature ramping time period; holding the sample at the second temperature (e.g., BHCT) and the selected pressure for a holding period; turning off the rotation for a shutdown time from t_{sd,start} to t_{sd,end}; turning the rotation back on at t_{sd,end} (e.g., after the shutdown time); and continuing to measure the Bc vs. time of the sample until the Bc value reaches 70, at time t_{TT}; and continuously recording the consistency (or “Bc”) reading (e.g., also known torque value) from the starting of the rotation of the cup at time to until time t_{TT}.

A twenty fifth embodiment can include the method of any one of the sixteenth to twenty fourth embodiments further comprising utilizing the consistency model during a cementing operation with a cement slurry having the same composition as the cement slurry design to predict Bc(t) (e.g., and thus provide an actual time t_{TT} at which the cement slurry will reach 70 Bc) during the cementing operation.

A twenty sixth embodiment can include the method of the twenty fifth embodiment, wherein utilizing the consistency model during the cementing operation with the cement slurry to predict the actual time t_{TT} at which the cement slurry will reach 70 Bc during the cementing operation further comprises: adjusting a time, a temperature, a consistency reading, or a combination thereof utilized by the consistency model with an actual time, an actual temperature, an actual consistency reading, or a combination thereof obtained during the cementing operation when the actual time, the actual temperature, the actual consistency reading, or the combination thereof obtained during the cementing operation is different from the time, the temperature, the consistency reading, or the combination thereof utilized to determine the parameters for the consistency model.

A twenty seventh embodiment can include the method of the twenty fifth or twenty sixth embodiment further comprising utilizing the Bc(t) predicted during the cementing operation to determine how one or more rheological properties of the cement slurry will change over time during the cementing operation.

A twenty eighth embodiment can include the method of the twenty seventh embodiment further comprising utilizing the determined changes in the one or more rheological properties with time to determine a hydraulic parameter of the cementing operation.

A twenty ninth embodiment can include the method of the twenty eighth embodiment, wherein the hydraulic parameter comprises an equivalent circulation density (ECD), a surface pressure, or a combination thereof.

A thirtieth embodiment can include the method of any one of the twenty seventh to twenty ninth embodiments, wherein utilizing the Bc(t) predicted during the cementing operation to determine how the one or more rheological properties of the cement slurry will change over time during the cementing operation further comprises: performing a rheology test at a reference temperature (e.g., a surface temperature, a mid temperature, and/or BHCT) to measure rheology data; determining GHB parameters based on the measured rheology data; calculating GHB parameters as a function of time based on the predicted Bc(t) during the cementing operation, applying the Bc-to-rheology model; and utilizing the GHB parameters as the function of time in a hydraulic calculation.

A thirty first embodiment can include the method of the thirtieth embodiment, wherein performing the rheology test at the reference temperature (e.g., surface temperature, a middle point temperature, and/or BHCT) to measure rheology data comprises conducting the rheology test using a rotational viscometer in accordance with API RP 10B-2 at the reference temperature.

A thirty second embodiment can include the method of the thirtieth or thirty first embodiment, wherein utilizing the Bc(t) predicted during the cementing operation to determine how the one or more rheological properties of the cement slurry will change over time during the cementing operation further comprises: for the cement slurry, calculating the rheological parameters (tau₀ (τ₀), mu_{∞}, m, and n, e.g., at 80° F.) applying a GHB rheological model (e.g., as displayed in Equation (1)); from Bc(t), from time t₀ until Bc reaches 70 at t_{TT}, calculating adjusted rheological parameters by computing tau₀ and mu_{∞} as a function of time, while m and n remain constant; and utilizing the adjusted rheological parameters for subsequent hydraulic calculations.

A thirty third embodiment can include the method of the thirty second embodiment, wherein: calculating the rheological parameters (tau₀ (τ₀), mu_{∞}, m, and n, e.g., at 80° F.) applying the GHB rheological model comprises using the Equation (1):

$${( \frac{τ}{{τ}_{ref}} )}^{m} = {( \frac{{τ}_{o}}{{τ}_{ref}} )}^{m} + {( \frac{{μ}_{∞} \dot{γ}}{{τ}_{ref}} )}^{n} \qquad Equation ( 1 )$$

wherein, for Bc vs. time data, until 70Bc, computing tau₀ and mu_{∞}, while m and n remain constant comprises using Equation (2) and Equation (3):

$${( \frac{{τ}_{o , i + 1}}{{τ}_{o , i}} )}^{m} = \frac{B {c}_{i + 1}}{B {c}_{i}} \qquad Equation ( 2 )$$

$${( \frac{{u}_{∞ , i + 1}}{{u}_{∞ , i}} )}^{n} = \frac{B {c}_{i + 1}}{B {c}_{i}} . \qquad Equation ( 3 )$$

A thirty fourth embodiment can include the method of the thirty second or thirty third embodiment, wherein utilizing the adjusted rheological parameters for subsequent hydraulic calculations further comprises: computing an adjusted viscosity using adjusted tao₀ and mu_{∞,0}; and using adjusted values of tao₀ vs. time and tao₀ and mu_{∞}, vs. time and m and n along with other job parameters (e.g., including fluid density, rate, and/or well bore geometry) to compute hydraulic friction based on real time events during the job.

In a thirty fifth embodiment, a method of designing a cement placement job comprises: predicting a hydraulic parameter of a cementing operation utilizing adjusted time dependent rheological model parameters, obtaining a time dependent rheological model parameter using a predicted time dependent Bc curve, and predicting the time dependent Bc curve using models that describe at least one of dissolution, thermal thinning, static gelling, and hydration processes in cement.

A thirty sixth embodiment can include the system or method of any previous embodiment further comprising utilizing tested Bc(t) data in place of or in addition to the predicted Bc(t) from the consistency model to calculate a hydraulic parameter.

A thirty seventh embodiment can include the system or method of any previous embodiment, wherein time dependent GHB parameters for use in hydraulics calculation(s) are calculated by other means alternatively or in addition to those calculated according to the fifth embodiment.

A thirty eighth embodiment can include the system or method of any previous embodiment, wherein one or more modules or process steps associated therewith are executed on a computer such as that shown and described with reference to FIG. 8.

While embodiments of the disclosure have been shown and described, modifications thereof can be made by one skilled in the art without departing from the spirit and teachings of the disclosure. The embodiments described herein are exemplary only, and are not intended to be limiting. Many variations and modifications of the disclosure disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L}, and an upper limit, R_{U}, is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. When a feature is described as “optional,” both embodiments with this feature and embodiments without this feature are disclosed. Similarly, the present disclosure contemplates embodiments where this feature is required and embodiments where this feature is specifically excluded. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the embodiments of the present disclosure.

What is claimed is:

1. A method comprising:
providing a consistency model that predicts consistency of a cement slurry design as a function of time (Bc(t));
performing a cementing operation with a cement slurry having a same composition as that of the cement slurry design; and
utilizing the consistency model to predict the Bc(t) of the cement slurry during the cementing operation.

2. The method of claim 1, wherein providing the consistency model comprises:
obtaining consistency versus time (Bc(t)) data for a sample of the cement slurry design from an initial time (t₀) of mixing sample of the cement slurry design to a thickening time (t_{TT}) at which the consistency of the sample reaches 70 Bc (Beardon consistency units); and
utilizing the consistency versus time data to determine coefficients utilized by the consistency model to predict the consistency of the cement slurry design as a function of time, wherein utilizing the consistency versus time data to determine the coefficients comprises statistical regression until a predicted Bc(t) simulates/models the Bc(t) data within a specified margin of error.

3. The method of claim 2, wherein the consistency model predicts the consistency as a function of time (Bc(t)) via:
a dissolution module [DM] that models the consistency behavior during dissolution of particles therein from the initial time (t₀) to a dissolution time (t_{d}) at an end of dissolution;
a thermal thinning module [TTM] that models thermal thinning, if present, during initial ramping of the temperature;
a static gelling module [SGM] that models consistency behavior of the cement slurry during a shut down, if present, from an initial shutdown time (t_{sd, start}) to an end of the shutdown (t_{sd, end}); and
a hydration module [HM] that models the consistency behavior during hydration of cementitious particles from an initial hydration time (tₕₒ) to the thickening time (t_{TT}), wherein
Bc(t)=Bc(i)[DM][TTM][SGM][HM],
wherein Bc(i) is the initial consistency at t₀.

4. A system comprising:
a processor;
a memory; and
an analysis program stored in the memory, wherein the analysis program is configured, when executed on the processor, to:
receive, during a cementing operation with a cement slurry, one or more adjusted parameters via a user interface;
use the one or more adjusted parameters in a consistency model; and
generate an updated consistency vs. time curve predicted by the consistency model, wherein the consistency model predicts the consistency of the cement slurry as a function of time (Bc(t)) via:
a dissolution module [DM] that models the consistency behavior of the cement slurry during dissolution of particles therein from the initial time (to) to a dissolution time (t_{d}) at an end of dissolution;
a thermal thinning module [TTM] that models thermal thinning of the cement slurry;
a static gelling module [SGM] that models consistency behavior of the cement slurry during shut down from an initial shutdown time (t_{sd, start}) to an end of the shutdown (t_{sd, end}); and
a hydration module [HM] that models the consistency behavior of the cement slurry during hydration of cementitious particles therein from an initial hydration time (tₕₒ) to the thickening time (t_{TT}), wherein
Bc(t)=Bc(i)[DM][TTM][SGM][HM],
wherein Bc(i) is the initial consistency of the cement slurry at t₀.

5. The system of claim 4, wherein the analysis program is further configured to:
receive rheology data of the cement slurry at one or more temperatures;
determine GHB parameters based on the rheology data; and
calculate adjusted GHB parameters as a function of time.

6. The system of claim 5, wherein the memory further comprises a hydraulics program, wherein the hydraulics program is configured to, when executed on the processor:
utilize the adjusted GHB parameters to determine a hydraulic parameter of the cementing operation.

7. The system of claim 4, wherein the dissolution module [DM] is only active if t_{d}>0, wherein:
if t_{d}=0, [DM]=1.0,
when t<t_{d}, [DM]=e\hat{ }(k_{d}t/t_{d}),
when t≥t_{d}, [DM]=[DM] at t=t_{d},
wherein t₀ is the initial time when the cement slurry is mixed, and t_{d} is the time at the end of dissolution.

8. The system of claim 7, wherein the one or more adjusted parameters comprises k_{d}, t_{d}, or a combination thereof.

9. The system of claim 4, wherein:
$$[ TTM ] = e \hat{} ( ( ( Delta E ) / R ) ( ( 1 / T ) - ( 1 / {T}_{i} ) ) ,$$
wherein Delta E is an activation energy for thermal thinning, R is the universal gas constant, Tᵢ is a temperature at the beginning of the TT test in Kelvin, and T is temperature at any given time t in Kelvin.

10. The system of claim 9, wherein the one or more adjusted parameters comprises Delta E, Tᵢ, or a combination thereof.

11. The system of claim 4, wherein:
[SGM] is only active if there is a shut down, and
when t≥t_{sd}, [SGM]=e\hat{ }((k_{sd}(t_{sd,end}−t_{sd,start}))),
wherein t_{sd,start} is a time at which shut down starts, t_{sd,end} is a time at shut down ends, and k_{sd} is a coefficient of static gelling reaction.

12. The system of claim 11, wherein the one or more adjusted parameters comprises an indication of whether or not there is a shut down, a start time of a shut down during the cementing operation (t_{sd,start/actual}), an end time of the shut down during the cementing operation (t_{sd,end/actual}), or a combination thereof.

13. The system of claim 4, wherein:
[HM]=e\hat{ }((k_{hyd})(t−tₕ₀)^{α}) from t=tₕ₀ to t=t_{TT},
wherein t_{TT}=thickening time at 70Bc, tₕₒ is a time when hydration begins, t=time, α (alpha_hyd) is a pseudo reaction order coefficient, and k_{hyd} is a hydration coefficient.

14. The system of claim 13, wherein the one or more adjusted parameters comprise tₕ₀.

15. A method comprising:
obtaining consistency versus time (Bc(t)) data from a thickening time (TT) test for a cement slurry design from an initial time (t₀) of mixing the cement slurry design to a thickening time (t_{TT}) at which the consistency reaches 70 Bc (Beardon consistency units); and
utilizing the consistency versus time data to determine parameters for a consistency model to predict the consistency of the cement slurry design as a function of time.

16. The method of claim 15, wherein the consistency model predicts the consistency of the cement slurry design as a function of time (Bc(t)) via:
a dissolution module [DM] that models the consistency behavior of the cement slurry design during dissolution of particles therein from the initial time (to) to a dissolution time (t_{d}) at an end of dissolution;
a thermal thinning module [TTM] that models thermal thinning, if present, of the cement slurry design from during initial ramping of the temperature during the obtaining of the consistency versus time (Bc(t)) data for the cement slurry design;
a static gelling module [SGM] that models consistency behavior of the cement slurry design during a shut down from a start of the shutdown (t_{sd, start}) to an end of the shutdown (t_{sd, end}); and
a hydration module [HM] that models the consistency behavior of the cement slurry design during hydration of cementitious particles therein from an initial hydration time (tₕₒ) to the thickening time (t_{TT}) at which the consistency reaches 70 Bc, wherein
Bc(t)=Bc(i)[DM][TTM][SGM][HM],
wherein Bc(i) is the initial consistency of the cement slurry deign at t₀.

17. The method of claim 16, wherein the dissolution module [DM] is only active if t_{d}>0, wherein:
if t_{d}=0, [DM]=1.0,
when t<t_{d}, [DM]=e\hat{ }(k_{d}t/t_{d}),
when t≥t_{d}, [DM]=[DM] at t=t_{d},
wherein t₀ is the initial time of mixing, at the beginning of the TT test, Bcᵢ is the initial consistency at the beginning of the TT test, and t_{d} is the dissolution time at an end of dissolution.

18. The method of claim 16, wherein:
$$[ TTM ] = e \hat{} ( ( ( Delta E ) / R ) ( ( 1 / T ) - ( 1 / {T}_{i} ) ) ,$$
wherein Delta E is an activation energy for thermal thinning, R is the universal gas constant, Tᵢ is a temperature at the beginning of the TT test in Kelvin, and T is temperature at any given time t in Kelvin.

19. The method of claim 16, wherein:
[SGM] module is only active if there is a shut down, and
when t≥t_{sd}, [SGM]=e\hat{ }((k_{sd}(t_{sd,end}−t_{sd,start}))),
wherein t_{sd,start} is a time at which rotation speed is stopped at the start of shut down, t_{sd,end} is a time at which rotation speed is resumed after the shut down, and k_{sd} is a coefficient of static gelling reaction.

20. The method of claim 16, wherein:
[HM]=e\hat{ }((k_{hyd})(t−tₕ₀)^{α}) from t=tₕ₀ to t=t_{TT},
wherein t_{TT}=thickening time at 70Bc, tₕₒ is a time when hydration begins, t=time, α is a pseudo reaction order coefficient, and k_{hyd} is a hydration coefficient.

21. A method of designing a cement placement job, the method comprising: predicting a hydraulic parameter of a cementing operation utilizing adjusted time dependent rheological model parameters, obtaining a time dependent rheological model parameter using a predicted time dependent Bc curve, and predicting the time dependent Bc curve using models that describe at least one of a parameter selected from the group consisting of dissolution, thermal thinning, static gelling, and hydration processes in cement.
